(19) United States
(12) Patent Application Publication
(10) Pub. No.: US 2021/0106621 A1
(43) Pub. Date: Apr. 15, 2021

(54) METHOD OF TREATING IMMUNOTHERAPY NON-RESPONDERS WITH AN AUTOLOGOUS CELL THERAPY

(71) Applicant: PACT Pharma, Inc., South San Francisco, CA (US)

(72) Inventors: Barbara Sennino, San Francisco, CA (US); Songming Peng, Hubei Province (CN); Stefanie Mandl-Cashman, San Francisco, CA (US); Alex Franzusoff, El Granada, CA (US)

(73) Assignee: PACT Pharma, Inc., South San Francisco, CA (US)

(21) Appl. No.: 17/067,101

(22) Filed: Oct. 9, 2020

Related U.S. Application Data
(60) Provisional application No. 63/024,909, filed on May 14, 2020.
(60) Provisional application No. 62/913,630, filed on Oct. 10, 2019.

(51) Int. Cl. A61K 35/17; C07K 14/725; C07K 14/73; C07K 14/705; C12N 15/63

(52) U.S. Cl. CPC A61K 35/17; C07K 14/7051; C12N 15/63; C07K 14/70517; C07K 14/70514

(57) ABSTRACT

Methods of treating cancer in Non-Responder Patients with an engineered NeoTCR Product are described herein.

### CROSS-REFERENCE TO RELATED APPLICATIONS

This application claims the benefit of priority of U.S. Provisional Application No. 62/913,630, filed Oct. 10, 2019, and U.S. Provisional Application No. 63/024,909, filed May 14, 2020, each of which is incorporated by reference herein in its entirety for any purpose.

### FIELD

Methods of treating cancer are provided, comprising administering to a subject with cancer CD8+ and/or CD4+ T cells engineered to express a neoepitope-specific T cell receptor. In some embodiments, the subject is an immunotherapy non-responder.

### BACKGROUND OF THE INVENTION

Cancer and progression of cancer are associated with immune suppression. In fact, cancer cells can activate immune checkpoint pathways in order to elicit immunosuppressive functions. Checkpoint inhibitors have thus been developed to reinvigorate the anti-tumor immune responses in patients by interrupting coinhibitory signaling pathways and promoting immune-mediated killing of cancer cells. Checkpoint inhibitors that target immune checkpoints provide promise for a subset of cancer patients who respond to such therapies. However, mainstream use of checkpoint inhibitors to treat all cancer patients has not been adopted because of the low response rate (including primary and secondary resistance) and immune-related adverse events in a large subset of such patients. In fact, approximately 60-70% of tumors are not responsive to single-agent checkpoint inhibitors and patients who do have tumors that respond to checkpoint inhibitors become resistant over time (Yan et al., Front Immunol. 208; 9:1739).

In addition to checkpoint inhibitors, cancer immunotherapies on a broader sense also succumb to the limitations of checkpoint inhibitors—many patients do not respond to current immunotherapies, and most that do eventually relapse. Furthermore, many patients experience adverse events as a result of the immunotherapies. For example, cancer vaccines have been explored as a potential immunotherapy; however, there have been few developments in the space of cancer vaccines that provide effective means of treating tumors without acquired resistance and/or toxicity. Another major limitation of immunotherapies is the unavailability of biomarkers to predict response rate for patients and to guide optimization of improvements to the existing immunotherapies to increase efficacy, decrease acquired and primary resistance, and decrease adverse events.

In infectious disease, polyclonal T cell responses against immunodominant epitopes drive successful immune responses. In cancer, neoepitopes derived from non-synonymous mutations, similarly to the immunodominant epitopes in viral infections, are potentially highly immunogenic because the T cells recognizing these antigens are not subjected to the mechanisms of central and peripheral tolerance. Indeed, early studies support that neoepitopes derived from non-synonymous mutations are the primary target of T cell responses induced by checkpoint inhibitor therapy and have been successfully targeted by adoptively transferred T cell therapies in multiple cancer histologies. However, there is limited knowledge on the immunodominance and evolution of neoepitopes, or the clonality of the T cell responses against these neoepitopes. Furthermore, little is known regarding the correlation between the presence and expansion of neoepitopes-specific T cells and the clinical response to checkpoint inhibitor therapy in patients.

Accordingly, there is a need to develop a therapy to treat cancer for patients who do not respond to checkpoint inhibitors and other existing immunotherapies. Disclosed herein is a therapy and methods of using such therapy for the treatment of cancer in patients who do not respond to checkpoint inhibitors and other immunotherapies.

### SUMMARY OF THE INVENTION

The present inventions described herein provide for methods of Non-Responder Patients in need thereof with a NeoTCR Product.
Embodiment 1. A method of treating cancer in a Non-Responder Patient with a NeoTCR Product.
Embodiment 2. A method of killing cancer cells from a Non-Responder Patient with a NeoTCR Product designed and made for such patient.
Embodiment 3. The method of embodiment 1 or 2, wherein the Non-Responder Patient does not respond to a checkpoint inhibitor therapy or an immunotherapy.
Embodiment 4. The method of embodiment 3, wherein the Non-Responder Patient showed no response to a checkpoint inhibitor therapy or an immunotherapy.
Embodiment 5. The method of embodiment 3, wherein the Non-Responder Patient initially responded to a checkpoint inhibitor therapy or an immunotherapy, then developed resistance, stopped responding, or showed reduced response.
Embodiment 6. The method of any one of embodiments 1-5, wherein the Non-Responder Patient did not respond to a CTLA-4 Binding Agent therapy.
Embodiment 7. The method of embodiment 6, wherein the CTLA-4 Binding Agent therapy is an anti-CTLA-4 antibody.
Embodiment 8. The method of embodiment 6 or embodiment 7, wherein the CTLA-4 Binding Agent therapy is ipilimumab.
Embodiment 9. The method of any one of embodiments 1-5, wherein the Non-Responder Patient did not respond to a PD-1 Axis Binding Agent therapy.
Embodiment 10. The method of embodiment 9, wherein the PD-1 Axis Binding Agent therapy is an anti-PD-1 antibody.
Embodiment 11. The method of embodiment 9 or embodiment 10, wherein the PD-1 Axis Binding Agent therapy is selected from pembrolizumab, nivolumab, cemiplimab, pidilizumab, and spartalizumab.
Embodiment 12. The method of embodiment 9, wherein the PD-1 Axis Binding Agent therapy is an anti-PD-L1 antibody.
Embodiment 13. The method of embodiment 9 or embodiment 12, wherein the PD-1 Axis Binding Agent therapy is selected from atezolizumab, avelumab, and durvalumab.
Embodiment 14. The method of any one of embodiments embodiment 1-5, wherein the Non-Responder Patient does not respond to an immunotherapy.
Embodiment 15. The method of embodiment 14, wherein the immunotherapy is a cancer vaccine therapy.
Embodiment 16. The method of embodiment 14 or embodiment 15, wherein the immunotherapy is T-VEC.
Embodiment 17. The method of any one of embodiments 1-16, wherein the NeoTCR Product comprises CD8+ and/or CD4+ T cells from the Non-Responder Patient, wherein the T cells have been engineered to express at least one neoTCR.
Embodiment 18. The method of embodiment 17, wherein the T cells in the NeoTCR Product have been engineered to express one, two, or three, neoTCRs.
Embodiment 19. The method of embodiment 18, wherein the T cells in the NeoTCR Product have been engineered to express one neoTCR.
Embodiment 20. The method of embodiment 18, wherein the T cells in the NeoTCR Product have been engineered to express a first neoTCR and a second neoTCR, wherein each T cell expresses either the first neoTCR or the second neoTCR.
Embodiment 21. The method of embodiment 18, wherein the T cells in the NeoTCR Product have been engineered to express a first neoTCR, a second neoTCR, and a third neoTCR, wherein each T cell expresses one neoTCR selected from the first neoTCR, the second neoTCR, and the third neoTCR.
Embodiment 22. The method of any one of embodiments 17-21, wherein each neoTCR binds a neoepitope comprising an amino acid mutation resulting from a somatic coding mutation present in the patient's cancer.
Embodiment 23. The method of any one of embodiments 17-22, wherein the neoTCR in the NeoTCR Product is present in the T cell genome at the endogenous TCR locus.
Embodiment 24. The method of any one of embodiments 1-23, wherein the NeoTCR Product is produced by a non-viral engineering method.
Embodiment 25. The method of any one of embodiments 1-24, wherein the NeoTCR Product is produced using CRISPR.
Embodiment 26. The method of any one of embodiments 17-25, wherein the NeoTCR Product does not comprise any exogenous DNA sequences in the genome of the T cells.
Embodiment 27. The method of any one of embodiments 17-26, wherein the T cells of the NeoTCR Product are derived from T cells from the patient.
Embodiment 28. A composition for the treatment of cancer in a Non-Responder Patient comprising a polynucleotide, wherein the polynucleotide comprises:
   a) first and second homology arms homologous to first and second target nucleic acid sequences;
   b) a TCR gene sequence positioned between the first and second homology arms;
   c) a first P2A-coding sequence positioned upstream of the TCR gene sequence and a second P2A-coding sequence positioned downstream of the TCR gene sequence, wherein the first and second P2A-coding sequences code for the same amino acid sequence that are codon-diverged relative to each other;
   d) a sequence coding for the amino acid sequence Gly Ser Gly positioned immediately upstream of the P2A-coding sequences; and
   e) a sequence coding for a Furin cleavage site positioned upstream of the second P2A-coding sequence.
Embodiment 29. The composition of embodiment 28, wherein the first and second homology arms of the polynucleotide are each from about 300 bases to about 2,000 bases in length.
Embodiment 30. The composition of embodiment 28 or 29, wherein the first and second homology arms of the polynucleotide are each about 600 bases to about 1,000 bases in length.
Embodiment 31. The composition of any of embodiments 28-30, wherein the polynucleotide further comprises a human growth hormone signal sequence positioned between the first P2A-coding sequence and the TCR gene sequence.
Embodiment 32. The composition of any of embodiments 28-31, wherein the polynucleotide further comprises a second TCR gene sequence positioned between the second P2A-coding sequence and the second homology arm.
Embodiment 33. The composition of embodiment 32, wherein the polynucleotide further comprises:
   a) a first human growth hormone signal sequence positioned between the first P2A-coding sequence and the first TCR gene sequence; and
   b) a second human growth hormone signal sequence positioned between the second P2A-coding sequence and the second TCR gene sequence,
   c) wherein the first and the second human growth hormone signal sequences are codon diverged relative to each other.
Embodiment 34. The composition of any of embodiments 28-33, wherein the polynucleotide further comprises an exogenous sequence of interest.
Embodiment 35. The composition of embodiment 34, wherein the exogenous sequence of interest encodes for a protein useful in autologous cell therapy.
Embodiment 36. The composition of any of embodiments 28-35, wherein the polynucleotide is a circular DNA.
Embodiment 37. The composition of any of embodiments 28-36, wherein the polynucleotide is a linear DNA.
Embodiment 38. The composition of any of embodiments 28-37, wherein the TCR gene sequence encodes for a TCR that recognizes a cancer antigen.
Embodiment 39. The composition of embodiment 38, wherein the cancer antigen is a neoantigen.
Embodiment 40. The composition of embodiment 38, wherein the cancer antigen is a patient specific neoantigen.
Embodiment 41. The composition of any of embodiments 28-40, wherein the TCR gene sequence is a patient specific TCR gene sequence.
Embodiment 42. The composition of any of embodiments 28-40, further comprising a nuclease.
Embodiment 43. The composition of embodiment 42, wherein the nuclease is a Clustered Regularly Interspaced Short Palindromic Repeats (CRISPR) family nuclease or derivative thereof.
Embodiment 44. The composition of embodiment 43, further comprising an sgRNA.
Embodiment 45. The composition of embodiment 42, wherein the nuclease targets an endogenous TCR locus.
Embodiment 46. The composition of embodiment 42, wherein the nuclease targets a TCR-alpha and a TCR-beta loci.
Embodiment 47. The composition of any of embodiments 28-47, wherein the first and second target nucleic acid sequences are positioned within an endogenous TCR locus.
Embodiment 48. The composition of embodiment 47, wherein the endogenous TCR locus is a TCR-alpha locus.
Embodiment 49. The composition of any of embodiments 28-48, wherein the polynucleotide is non-viral.
Embodiment 50. The composition of any of embodiments 28-49, wherein the polynucleotide is a gene therapy vector.
Embodiment 51. A composition for the treatment of cancer in a Non-Responder Patient comprising a polynucleotide, wherein the polynucleotide comprises:
   a) first and second homology arms homologous to first and second target nucleic acid sequences;
   b) a TCR gene sequence positioned between the first and second homology arms;
   c) a first P2A-coding sequence positioned upstream of the TCR gene sequence and a second P2A-coding sequence positioned downstream of the TCR gene sequence, wherein the first and second P2A-coding sequences code for the same amino acid sequence that are codon-diverged relative to each other;
   d) a sequence coding for a flexible linker positioned immediately upstream of the P2A-coding sequences; and
   e) a sequence coding for a protease cleavage sequence positioned upstream of the second P2A-coding sequence.
Embodiment 52. A composition for the treatment of cancer in a Non-Responder Patient comprising a circular polynucleotide, wherein the circular polynucleotide comprises:
   a) first and second homology arms homologous to first and second target nucleic acid sequences;
   b) a TCR gene sequence positioned between the first and second homology arms;
   c) a first P2A-coding sequence positioned upstream of the TCR gene sequence and a second P2A-coding sequence positioned downstream of the TCR gene sequence, wherein the first and second P2A-coding sequences code for the same amino acid sequence that are codon-diverged relative to each other;
   d) a sequence coding for the amino acid sequence Gly Ser Gly positioned immediately upstream of the P2A-coding sequences; and
   e) a sequence coding for a Furin cleavage site positioned upstream of the second P2A-coding sequence.
Embodiment 53. A method of treating a cancer in a Non-Responder Patient comprising:
   a) administering to the human patient a therapeutically effective population of modified primary cells, wherein the modified primary cells comprise an exogenous nuclease and a non-viral exogenous nucleic acid sequence incorporated into an endogenous locus, the non-viral exogenous nucleic acid comprising:
      i. a TCR gene sequence coding for at least a portion of a TCR capable of binding an antigen present on a cancerous cell,
      ii. a first P2A-coding sequence positioned upstream of the TCR gene sequence and a second P2A-coding sequence positioned downstream of the TCR gene sequence, wherein the first and second P2A-coding sequences code for the same amino acid sequence that are codon-diverged relative to each other,
      iii. a sequence coding for the amino acid sequence Gly Ser Gly positioned immediately upstream of the P2A-coding sequences;
      iv. a sequence coding for a Furin cleavage site positioned upstream of the second P2A-coding sequence; and

thereby treating the cancer in the human patient.
Embodiment 54. The method of embodiment 53, wherein the exogenous nuclease is a Clustered Regularly Interspaced Short Palindromic Repeats (CRISPR) family nuclease or derivative thereof.
Embodiment 55. The method of embodiment 53 or 54, wherein the exogenous nucleic acid comprises a second TCR gene sequence positioned immediately downstream of the second P2A-coding sequence.
Embodiment 56. The method of any of embodiments 53-55, wherein the exogenous nucleic acid further comprises a human growth hormone signal sequence positioned between the first P2A-coding sequence and the TCR gene sequence.
Embodiment 57. The method of embodiment 56, wherein the exogenous nucleic acid further comprises:
   a) a first human growth hormone signal sequence positioned between the first P2A-coding sequence and the first TCR gene sequence; and
   b) a second human growth hormone signal sequence positioned between the second P2A-coding sequence and the second TCR gene sequence;

wherein the first and the second human growth hormone signal sequences are codon diverged relative to each other.
Embodiment 58. The method of any of embodiments 53-57, wherein prior to administering to the human patient a therapeutically effective population of modified primary cells, a non-myeloablative lymphodepletion regimen is administered to the patient.
Embodiment 59. The method of any of embodiments 53-58, wherein the cancer is selected from melanoma, lung cancer, breast cancer, head and neck cancer, ovarian cancer, prostate, and colorectal cancer.
Embodiment 60. A method of treating a cancer in a Non-Responder Patient comprising:
   a) modifying a patient-derived T cell by introducing a nuclease-mediated introduction of a non-viral polynucleotide into the T cell, wherein the non-viral polynucleotide comprises:
      i. first and second homology arms homologous to first and second endogenous sequences of the cell;
      ii. a TCR gene sequence positioned between the first and second homology arms; and
      iii. a first P2A-coding sequence positioned upstream of the TCR gene sequence and a second P2A-coding sequence positioned downstream of the TCR gene sequence, wherein the first and second P2A-coding sequences code for the same amino acid sequence that are but codon-diverged relative to each other;
      iv. a sequence coding for the amino acid sequence Gly Ser Gly positioned immediately upstream of the P2A-coding sequences; and
      v. a sequence coding for a Furin cleavage site positioned upstream of the second P2A-coding sequence;
   b) recombining the non-viral polynucleotide into an endogenous locus of the T cell, wherein the endogenous locus comprises the first and second endogenous sequences homologous to the first and second homology arms of the non-viral polynucleotide; and
   c) culturing the modified T cell to produce a population of T cells; and
   d) administering a therapeutically effective number of the modified T cells to the human patient to thereby treat the cancer.
Embodiment 61. The method of embodiment 60, wherein said recombining further comprises
   a) cleavage of the endogenous locus by a nuclease; and
   b) recombination of the non-viral polynucleotide into the endogenous locus by homology directed repair.
Embodiment 62. The method of embodiment 60 or 61, wherein the non-viral polynucleotide further comprises a second TCR gene sequence positioned between the second P2A-coding sequence and the second homology arm.
Embodiment 63. The method of any of embodiments 60-62, wherein the first and second homology arms are each from about 300 bases to about 2,000 bases in length.
Embodiment 64. The method of any of embodiments 60-63, wherein the non-viral polynucleotide further comprises a human growth hormone signal sequence positioned between the first P2A-coding sequence and the TCR gene sequence.
Embodiment 65. The method of any of embodiments 60-64, wherein prior to administering a therapeutically effective number of modified T cells, a non-myeloablative lymphodepletion regimen is administered to the subject.
Embodiment 66. The method of any of embodiments 60-65, wherein the cancer is selected from melanoma, lung cancer, and colorectal cancer.
Embodiment 67. The method of any of embodiments 60-66, wherein the nuclease is a Clustered Regularly Interspaced Short Palindromic Repeats (CRISPR) family nuclease or derivative thereof.
Embodiment 68. The method of embodiment 67, wherein the non-viral polynucleotide further comprises:
   a) a first human growth hormone signal sequence positioned between the first P2A-coding sequence and the first TCR gene sequence; and
   b) a second human growth hormone signal sequence positioned between the second P2A-coding sequence and the second TCR gene sequence;

wherein the first and second human growth hormone sequences are codon diverged relative to each other.
Embodiment 69. The method of any of embodiments 60-68, wherein the patient-derived T cell has been frozen prior to the introduction of the polynucleotide.
Embodiment 70. The method of any of embodiments 60-69, wherein the first and second homology arms are each about 600 bases to about 1,000 bases in length.
Embodiment 71. Any method or composition described herein.

### BRIEF DESCRIPTOIN OF THE DRAWINGS

FIGS. 1A-1B show neoepitope-specific T-cell isolation and TCR sequencing. FIG. 1A, Schematic of the neoantigen-specific TCR isolation from patient samples. NeoE, neoepitope FIG. 1B, circle packing representing the number of non-synonymous mutations, mutations screened, putative neoepitope-HLA screened, mutations targeted by neoepitope-specific T cells, and neoepitope-specific T-cell clonotypes isolated in patients with (patient #1 and #2, also referred to as PT1 and PT2, respectively) or without (patient #3, #4, and #5, also referred to as PT3, PT4, and PT5, respectively) response to anti-PD-1 therapy.

FIGS. 2A-2D show neoepitope-specific T-cell isolation from tumor infiltrating lymphocytes (TILs) and PBMCs in patients with a response to anti-PD-1 therapy. FIG. 2A, tumor measurements over time and sample collection for patient #1. FIG. 2B, landscape analysis of the neoepitope-specific T cells over time in patient #1. Bottom panel shows mRNA expression and predicted HLA binding affinity of the neoantigens screened. Neoepitopes are highlighted in different patterned bars, and the mutated gene name, the sequence of the neoepitope with the point mutation shown with the point mutation underlined, and the HLA are indicated on top. The same pattern code is used in the top panels to show the neoepitope specificity of the isolated T cells. The five top panels show the evolution over time of the neoepitope-specific T cells in TILs and PBMCs. Each little box represent one isolated T cell and a cross is equivalent to ten isolated T cells. Each dotted line box represents a different neoepitope-specific T-cell clonotype. The number of isolated T cells is normalized to 100,000 CD8+ T cells using a round up method to plot. Only validated T-cell clonotypes are shown. FIG. 2C, same as a for patient #2. This patient had two target lesions; the average of the longest diameters is shown. FIG. 2D, same as FIG. 2B for patient #2.

FIGS. 3A-3E show captured neoTCR validation. After capture of the neoepitope-specific T cells, the cognate TCR is sequenced and the sequence used to gene edit healthy donor T cells replacing the endogenous TCR by the neoTCR. The neoTCR specificity and stability are validated by dextramer staining of the gene edited T cells. Only validated TCRs are shown. FIGS. 3A-3E show dextramer staining of the gene edited T-cell products from patients #1-#5 respectively. FIG. 3A shows patient 1, FIG. 3B shows patient 2, FIG. 3C shows patient 3, FIG. 3D shows patient 4, and FIG. 3E shows patient 5.

FIGS. 4A-4I show neoepitope-specific T-cell isolation from TILs and PBMCs and neoTCR anti-tumor activity in patients without a response to anti-PD-1. FIGS. 4A-4C, landscape of the neoepitope-specific T cells for patients #3, #4 and #5 respectively. Bottom panel shows mRNA expression and predicted HLA binding affinity of the neoantigens screened. Neoepitopes are highlighted in different patterns, the mutated gene name, the sequence of the neoepitope with the point mutation marked in red, and the HLA are indicated on top. Predicted HLA-binding affinity for the neoepitope in patient #5 is highlighted with an arrow, since the expression is considered negative. The same pattern code is used on the top panels to show the neoepitope specificity of the isolated T cells. Each little box represents one isolated T cell. Each color represents a different neoepitope-specific T-cell clonotype. The number of isolated T cells is normalized to 100,000 CD8+ T cells using a round up method to plot. Only validated T-cell clonotypes are shown. FIGS. 4D-4F, 4-1BB upregulation in the CD8+ neoTCR+ gene edited T cells from patients #3, #4 and #5 respectively, upon co-culture with the autologous (M485, M486 and M488, respectively) or mismatched (M202) cell lines (n=3). FIGS. 4G-4I, specific target cell killing by neoTCR gene edited T cells from patients #3, #4 and #5 respectively in the autologous cell line and the mismatched control (P:T) ratio 5:1, n=4 for patient #3 and #5, P:T ratio 10:1, n=3 for patient #4). *p<0.05, **p<0.005, ***p<0.0005, ****p<0.0001 vs Neo12, unpaired t test with Holm-Sidak adjustment for multiple comparison in FIGS. 4D, 4E, 4G, and 4H. The same test without adjustment for multiple comparisons was used in FIGS. 4F and 4I. (n) indicates the number of biological replicates. Mean±SD is shown. All T-cell products contain CD8+ and CD4+ gene edited T cells.

FIGS. 5A-5D show anti-tumor activity of the neoepitope-specific TCRs isolated in patients with response to anti-PD-1. Healthy donor T cells were genetically modified to replace the endogenous TCR by the isolated neoTCRs from patient #1 (FIGS. 5A and 5B), and patient #2 (FIGS. 5C and 5D) and used to characterize the anti-tumor activity of these neoTCRs. FIG. 5A, 4-1BB upregulation in the CD8+ neoTCR+ gene edited T cells from patient #1 upon co-culture with the autologous (M489) or mismatched (M202) cell lines (n=3). FIG. 5B, specific target cell killing by neoTCR gene edited T cells from patient #1 in the autologous cell line (M489) and the mismatched control (M202) (product: target-P:T-ratio 1:1, n=4). FIG. 5C, 4-1BB upregulation in CD8+ neoTCR+ gene edited T cells from patient #2 upon co-culture with the autologous (M490) or mismatched (M202) cell lines pre-treated for 24 h with IFNγ (n=3). FIG. 5D, specific target cell killing by neoTCR gene edited T cells in the autologous cell line (M490) and the mismatched control (M202), target cells pre-treated for 24 h with IFNγ (P:T ratio 10:1, n=4). *p<0.05, **p<0.005, ***p<0.0005, ****p<0.0001 vs Neo12, unpaired t test with Holm-Sidak adjustment for multiple comparisons. (n) indicates the number of biological replicates. Mean±SD is shown. All T-cell products contain CD8+ and CD4+ gene edited T cells.

FIGS. 6A-6D show activation, cytokine secretion, cytotoxicity, and proliferation induced by neoepitope-specific TCRs from patient #1 upon co-culture with the autologous cell line. Healthy donor T cells genetically engineered to express the captured neoTCRs from patient #1 were co-cultured with the autologous (M489) or a mismatched cell line (M202). FIG. 6A, 4-1BB, OX-40, and CD107a upregulation in the CD8+ neoTCR+ T cells after co-culture. Melanoma cell lines were pre-treated with regular media or media with IFNγ 24 h prior co-culture with T cells (n=3). The graphs in FIG. 6A have four-plex plots of M202−IFNγ, M202+IGNγ, M489−IFNγ, and M489+IFNγ (shown in that order with the M489−IFNγ and M489+IFNγ being the right two bars in that order in each fourplex). FIG. 6B, cytokine release at 24 h after co-culture (n=3). FIG. 6C, % tumor growth inhibition compared to the cell growth in media alone at 24, 48, 72 and 96 h (n=4). FIG. 6D, proliferation of CD8+ neoTCR+ T cells measured by Ki67 mean fluorescence intensity upon 24, 48 and 72 h co-culture with autologous melanoma cell line (M489, top panel) or a mismatched cell line (M202, bottom panel) (n=3). Each set of hourly measurements are shown as triplex of bars. The order of the bars from left to right are 24 hr, 48 hr, and 72 hr. *p<0.05, **p<0.005, ***p<0.0005, ****p<0.0001 vs Neo12, unpaired t test with Holm-Sidak adjustment for multiple comparisons in figure a, b and c. *p<0.05, **p<0.005, ***p<0.0005, ****p<0.0001 vs M202, unpaired t test with Holm-Sidak adjustment for multiple comparisons in FIG. 6D. (n) indicates the number of biological replicates. Mean±SD is shown. All T-cell products contain CD8+ and CD4+ gene edited T cells.

FIGS. 7A-7D show activation, cytokine secretion, cytotoxicity, and proliferation induced by neoepitope-specific TCRs from patient #2 upon co-culture with the autologous cell line. Healthy donor T cells genetically engineered to express the captured neoTCRs from patient #2 were co-cultured with the autologous (M490) or a mismatched cell line (M202). FIG. 7A, 4-1BB and OX-40 upregulation in the CD8+ neoTCR+ T cells after co-culture. Melanoma cell lines were pre-treated with regular media or media with IFNγ 24 h prior co-culture with T cells (n=3). The graphs in FIG. 7A have four-plex plots of M202−IFNγ, M202+IGNγ, M489−IFNγ, and M489+IFNγ (shown in that order with the M489−IFNγ and M489+IFNγ being the right two bars in that order in each fourplex). FIG. 7B, specific target cell killing in the autologous cell line (top panel) or a mismatched cell line (bottom panel), (P:T ratio 10:1, n=4). FIG. 7C, cytokine release at 24 h after co-culture (n=3). Melanoma cell lines were pre-treated with IFNγ for 24 h before co-culture with T cells. d, Proliferation of CD8+ neoTCR+T cells measured by Ki67 mean fluorescence intensity upon 24, 48 and 72 h co-culture with autologous melanoma cell line (M490, top panel) or a mismatched cell line (M202, bottom panel) (n=3). *p<0.05, **p<0.005, ***p<0.0005, ****p<0.0001 vs Neo12, unpaired t test with Holm-Sidak adjustment for multiple comparisons in FIGS. 7A-7C. *p<0.05, **p<0.005, ***p<0.0005, ****p<0.0001 vs M202, unpaired t test with Holm-Sidak adjustment for multiple comparisons in FIG. 7D. Each set of hourly measurements in FIG. 7D are shown as triplex of bars; the order of the bars from left to right are 24 hr, 48 hr, and 72 hr. (n) indicates the number of biological replicates. Mean±SD is shown. All T-cell products contain CD8+ and CD4+ gene edited T cells.

FIGS. 8A-8F show activation, cytokine secretion, and proliferation induced by neoepitope-specific TCRs from patient without a response to anti-PD-1. Healthy donor T cells genetically engineered to express the captured neoTCRs from patients #3 (FIG. 8A-8C), #4 (FIGS. 8D-8E) and #5 (FIG. 7F) were co-cultured with the autologous (M485, M468 and M488 respectively) or a mismatched cell line (M202). FIG. 8A, 4-1BB, OX-40, and CD107a upregulation in the CD8+ neoTCR+ T cells from patient #3 after co-culture. Melanoma cell lines were pre-treated with regular media or media with IFNγ 24 h prior co-culture with T cells (n=3). The graphs in FIG. 8A have four-plex plots of M202−IFNγ, M202+IGNγ, M489−IFNγ, and M489+IFNγ (shown in that order with the M489−IFNγ and M489+IFNγ being the right two bars in that order in each fourplex). FIG. 8B, cytokine release at 24 h after co-culture (n=3). FIG. 8C, proliferation of CD8+ neoTCR+ T cells from patient #3 measured by Ki67 mean fluorescence intensity upon 24, 48 and 72 h co-culture with autologous melanoma cell line (M485, top panel) or a mismatched cell line (M202, bottom panel) (n=3). Each set of hourly measurements in FIG. 8C are shown as triplex of bars; the order of the bars from left to right are 24 hr, 48 hr, and 72 hr. FIG. 8D, 4-1BB and OX-40 upregulation in the CD8+ neoTCR+ T cells from patient #4 after co-culture. Melanoma cell lines were pre-treated with regular media or media with IFNγ 24 h prior co-culture with T cells (n=3). The graphs in FIG. 8D have four-plex plots of M202−IFNγ, M202+IGNγ, M489−IFNγ, and M489+IFNγ (shown in that order with the M489−IFNγ and M489+IFNγ being the right two bars in that order in each fourplex). FIG. 8E, cytokine release at 24 h after co-culture (n=3).). FIG. 8F, 4-1BB upregulation in the CD8+ neoTCR+ T cells from patient #5 after co-culture. Melanoma cell lines were pre-treated with regular media or media with IFNγ 24 h prior co-culture with T cells (n=3). *p<0.05, **p<0.005, ***p<0.0005, ****p<0.0001 vs Neo12, unpaired t test with Holm-Sidak adjustment for multiple comparisons in FIGS. 8A, 8B, 8D, 8E, and 8F. The graphs in FIG. 8F have four-plex plots of M202−IFNγ, M202+IGNγ, M489−IFNγ, and M489+IFNγ (shown in that order with the M489−IFNγ and M489+IFNγ being the right two bars in that order in each fourplex). *p<0.05, **p<0.005, ***p<0.0005, ****p<0.0001 vs M202, unpaired t test with Holm-Sidak adjustment for multiple comparisons in figure c. (n) indicates the number of biological replicates. Mean±SD is shown. All T-cell products contain CD8+ and CD4+ gene edited T cells.

FIG. 9 shows that the NeoTCR Product designed for a specific Non-Responder Patient causes cancer cell death (see top three photographs of cultured cancer cells). The top three photographs show the Non-Responder Patient's cancer cells in culture at Day 0 and at Days 2 and 5 after administration of the NeoTCR Product designed specifically for the patient. Cancer cell death is visibly apparent.

FIG. 10 shows that neoepitopes can be detected in Non-Responder Patients. Based on the detected neoepitopes, NeoTCR Products can be individually designed and made for each Non-Responder Patient.

FIG. 11 shows that tumor cells from Non-Responder Patients (the “autologous tumor cells” as shown in the legend), when exposed to a negative control (the “Neo12”) does not elicit upregulation of anti-tumor factors/signals (4-1BB, Ox40, Ki67, INFγ, TNFα, and CD107). However, when the Non-Responder Patients were exposed to NeoTCR Products designed based on their tumor neoantigens (TCR36, referred to as “TCR212” and TCR37, referred to as “TCR213”), anti-tumor factors/signals were upregulated and expressed. Tumor cells from a different patient (mis-matched with the TCR212 and TCR213 NeoTCR Products) served as an additional control and showed no upregulation of anti-tumor factors/signals.

FIGS. 12A-12C show an example of a NeoE TCR cassette and gene editing methods that can be used to make NeoTCR Products. FIG. 12A shows a schematic representing the general targeting strategy used for integrating neoantigen-specific TCR constructs (neoTCRs) into the TCRα locus. FIGS. 12B and 12C show a neoantigen-specific TCR construct design used for integrating a NeoTCR into the TCRα locus wherein the cassette is shown with signal sequences (“SS”), protease cleavage sites (“P”), and 2A peptides (“2A”). FIG. 12B shows a target TCRα locus (endogenous TRAC, top panel) and its CRISPR Cas9 target site (horizontal stripes, cleavage site designated by the arrow), and the circular plasmid HR template (bottom panel) with the polynucleotide encoding the neoTCR, which is located between left and right homology arms (“LHA” and “RHA” respectively) prior to integration. FIG. 12C shows the integrated neoTCR in the TCRα locus (top panel), the transcribed and spliced neoTCR mRNA (middle panel), and translation and processing of the expressed neoTCR (bottom panel).

### DETAILED DESCRIPTION OF THE INVENTION

The present disclosure is based on the discovery that NeoTCR Products can be used to treat Non-Responder Patients.

### Definitions

Unless defined otherwise, all technical and scientific terms used herein have the meaning commonly understood by a person skilled in the art. The following references provide one of skill with a general definition of many of the terms used in the presently disclosed subject matter: Singleton et al., Dictionary of Microbiology and Molecular Biology (2nd ed. 1994); The Cambridge Dictionary of Science and Technology (Walker ed., 1988); The Glossary of Genetics, 5th Ed., R. Rieger et al. (eds.), Springer Verlag (1991); and Hale & Marham, The Harper Collins Dictionary of Biology (1991). As used herein, the following terms have the meanings ascribed to them below, unless specified otherwise.

It is understood that aspects and embodiments of the invention described herein include ““comprising,”” ““consisting,”“and “”consisting essentially of”” aspects and embodiments. The terms ““comprises”” and “comprising” are intended to have the broad meaning ascribed to them in U.S. Patent Law and can mean “includes”, “including” and the like.

As used herein, the term “about” or “approximately” means within an acceptable error range for the particular value as determined by one of ordinary skill in the art, which will depend in part on how the value is measured or determined, i.e., the limitations of the measurement system. For example, “about” can mean within 3 or more than 3 standard deviations, per the practice in the art. Alternatively, “about” can mean a range of up to 20%, e.g., up to 10%, up to 5%, or up to 1% of a given value. Alternatively, particularly with respect to biological systems or processes, the term can mean within an order of magnitude, e.g., within 5-fold or within 2-fold, of a value.

“Checkpoint Inhibitor” as used herein means a type of drug that blocks certain proteins made by certain types of immune system cells (e.g., T cells) and a subset of cancer cells. Such proteins that are made by certain immune and cancer cells help keep immune responses in check and can keep T cells from killing cancer cells. Accordingly, when these proteins are blocked by a checkpoint inhibitor, T cells are able to kill certain cancer cells. A checkpoint inhibitor is an immunotherapy and the terms are not mutually exclusive as used herein.

The terms “Cancer” and “Tumor” are used interchangeably herein. As used herein, the terms “Cancer” or “Tumor” refer to all neoplastic cell growth and proliferation, whether malignant or benign, and all pre-cancerous and cancerous cells and tissues. The terms are further used to refer to or describe the physiological condition in mammals that is typically characterized by unregulated cell growth/proliferation. Cancer can affect a variety of cell types, tissues, or organs, including but not limited to an organ selected from bladder, bone, brain, breast, cartilage, glia, esophagus, fallopian tube, gallbladder, heart, intestines, kidney, liver, lung, lymph node, nervous tissue, ovaries, pancreas, prostate, skeletal muscle, skin, spinal cord, spleen, stomach, testes, thymus, thyroid, trachea, urogenital tract, ureter, urethra, uterus, and vagina, or a tissue or cell type thereof. Cancer includes cancers, such as sarcomas, carcinomas, or plasmacytomas (malignant tumor of the plasma cells). Examples of cancer include, but are not limited to, those described herein. The terms “Cancer” or “Tumor” and “Proliferative Disorder” are not mutually exclusive as used herein.

“CTLA-4 Binding Agent” as used herein means a molecule that binds CTLA-4 and decreases, blocks, inhibits, abrogates or interferes with signal transduction resulting from the interaction of PD-1 with one or more of its binding partners, such as B7-1 and/or B7-2. In some embodiments, the CTLA-4 binding agent is a molecule that inhibits the binding of CTLA-4 to one or more of its binding partners. In some embodiments, the CTLA-4 binding agent inhibits the binding of CTLA-4 to B7-1 and/or B7-2. For example, CTLA-4 binding agents include anti-CTLA-4 antibodies, antigen binding fragments thereof, immunoadhesins, fusion proteins, oligopeptides and other molecules that decrease, block, inhibit, abrogate or interfere with signal transduction resulting from the interaction of CTLA-4 with B7-1 and/or B7-2. In some embodiments, the CTLA-4 binding agent is an anti-CTLA-4 antibody. In a specific embodiment, a CTLA-4 binding agent is ipilimumab.

“Dextramer” as used herein means a multimerized neoepitope-HLA complex that specifically binds to its cognate NeoTCR.

“Endogenous” as used herein refers to a nucleic acid molecule or polypeptide that is normally expressed in a cell or tissue.

“Exogenous” as used herein refers to a nucleic acid molecule or polypeptide that is not endogenously present in a cell. The term “exogenous” would therefore encompass any recombinant nucleic acid molecule or polypeptide expressed in a cell, such as foreign, heterologous, and over-expressed nucleic acid molecules and polypeptides. By “exogenous” nucleic acid is meant a nucleic acid not present in a native wild-type cell; for example, an exogenous nucleic acid may vary from an endogenous counterpart by sequence, by position/location, or both. For clarity, an exogenous nucleic acid may have the same or different sequence relative to its native endogenous counterpart; it may be introduced by genetic engineering into the cell itself or a progenitor thereof, and may optionally be linked to alternative control sequences, such as a non-native promoter or secretory sequence.

“Immunotherapy” or “Cancer Immunotherapy” as used herein means a therapy designed to treat a disease such as cancer by activating or suppressing the immune system. Immunotherapies can be designed to elicit or amplify an immune response (i.e., activation immunotherapies) or to reduce or suppress an immune response (i.e., suppression immunotherapies). A checkpoint inhibitor is an immunotherapy and the terms are not mutually exclusive as used herein.

“Neoantigen”, “neoepitope” or “neoE” refer to a newly formed antigenic determinant that arises, e.g., from a somatic mutation(s) and is recognized as “non-self.” A mutation giving rise to a “neoantigen”, “neoepitope” or “neoE” can include a frameshift or non-frameshift indel, missense or nonsense substitution, splice site alteration (e.g., alternatively spliced transcripts), genomic rearrangement or gene fusion, any genomic or expression alterations, or any post-translational modifications

“NeoTCR” as used herein mean a neoepitope-specific T cell receptor that is introduced into a T cell, e.g., by gene editing methods. As used herein, the term “TCR gene sequence” refers to a NeoTCR gene sequence.

“NeoTCR cells” as used herein means one or more cells precision engineered to express one or more NeoTCRs. In certain embodiments, the cells are T cells. In certain embodiments, the T cells are CD8+ and/or CD4+ T cells. In certain embodiments, the CD8+ and/or CD4+ T cells are autologous cells from the patient for whom a NeoTCR Product will be administered. The terms “NeoTCR cells” and “NeoTCR-P1 T cells” and “NeoTCR-P1 cells” are used interchangeably herein. In some embodiments, the NeoTCR cells do not comprise any exogenous DNA sequences, e.g., in the genome of the T cells.

“NeoTCR Product” as used herein means a pharmaceutical formulation comprising one or more NeoTCR cells. NeoTCR Product consists of autologous precision genome-engineered CD8+ and CD4+ T cells. Using a targeted DNA-mediated non-viral precision genome engineering approach, expression of the endogenous TCR is eliminated and replaced by a patient-specific NeoTCR isolated from peripheral CD8+ T cells targeting the tumor-exclusive neoepitope. In certain embodiments, the resulting engineered CD8+ and/or CD4+ T cells express NeoTCRs on their surface of native sequence, native expression levels, and native TCR function. The sequences of the NeoTCR external binding domain and cytoplasmic signaling domains are unmodified from the TCR isolated from native CD8+ T cells. Regulation of the NeoTCR gene expression is driven by the native endogenous TCR promoter positioned upstream of where the NeoTCR gene cassette is integrated into the genome. Through this approach, native levels of NeoTCR expression are observed in unstimulated and antigen-activated T cell states. In some embodiments, the NeoTCR Product does not comprise any exogenous DNA sequences, e.g., in the genome of the T cells.

The NeoTCR Product manufactured for each patient represents a defined dose of autologous CD8+ and/or CD4+ T cells that are precision genome engineered to express a single neoepitope (neoE)-specific TCR cloned from neoE-specific CD8+ T cells individually isolated from the peripheral blood of that same patient.

“NeoTCR Viral Product” as used herein has the same definition of NeoTCR Product except that the genome engineering is performed using viral mediated methods.

“Non-Responder Patient” as used herein refers to a patient with cancer wherein the cancer does not respond to checkpoint inhibitors and/or immunotherapies either because they did not respond to the respective treatment or because they initially responded, but developed resistance, and stopped responding or showed reduced response, to the respective treatment over time. A Non-Responder Patient includes patients who have no response or only a partial response to a checkpoint inhibitor and/or immunotherapy.

The term “PD-1 axis binding agent” refers to a molecule that inhibits the interaction of a PD-1 axis binding partner with either one or more of its binding partner, so as to remove T cell dysfunction resulting from signaling on the PD-1 signaling axis—with a result being to restore or enhance T cell function (e.g., proliferation, cytokine production, target cell killing). As used herein, the term PD-1 axis binding agent includes PD-1 binding agents, PD-L 1 binding agents, and PD-L2 binding agents.

The term “PD-1 binding agent” refers to a molecule that binds PD-1 and decreases, blocks, inhibits, abrogates or interferes with signal transduction resulting from the interaction of PD-1 with one or more of its binding partners, such as PD-L1 and/or PD-L2. In some embodiments, the PD-1 binding agent is a molecule that inhibits the binding of PD-1 to one or more of its binding partners. In some embodiments, the PD-1 binding agent inhibits the binding of PD-1 to PD-L1 and/or PD-L2. For example, PD-1 binding agents include anti-PD-1 antibodies, antigen binding fragments thereof, immunoadhesins, fusion proteins, oligopeptides and other molecules that decrease, block, inhibit, abrogate or interfere with signal transduction resulting from the interaction of PD-1 with PD-L1 and/or PD-L2. In some embodiments, a PD-1 binding agent reduces the negative co-stimulatory signal mediated by or through cell surface proteins expressed on T lymphocytes mediated signaling through PD-1 so as render a dysfunctional T cell less dysfunctional (e.g., enhancing effector responses to antigen recognition). In some embodiments, the PD-1 binding agent is an anti-PD-1 antibody. In a specific embodiment, a PD-1 binding agent is nivolumab. In another specific embodiment, a PD-1 binding agent is pembrolizumab. In another specific embodiment, a PD-1 binding agent is cemiplimab. In another specific embodiment, a PD-1 binding agent is pidilizumab. In another specific embodiment, a PD-1 binding agent is AMP-224. In another specific embodiment, a PD-1 binding agent is MED1-0680 (Medimmune). In another specific embodiment, a PD-1 binding agent is spartalizumab (PDR001). In another specific embodiment, a PD-1 binding agent is REGN2810 (Regeneron). In another specific embodiment, a PD-1 binding agent is BGB-108 (BeiGene).

The term “PD-L1 binding agent” refers to a molecule that binds PD-L1 and decreases, blocks, inhibits, abrogates or interferes with signal transduction resulting from the interaction of PD-L1 with one or more of its binding partners, such as PD-1 and/or B7-1. In some embodiments, a PD-L1 binding agent is a molecule that inhibits the binding of PD-L1 to its binding partners. In some embodiments, the PD-L1 binding agent inhibits binding of PD-L1 to PD-1 and/or B7-1. In some embodiments, the PD-L1 binding agent include anti-PD-L1 antibodies, antigen binding fragments thereof, immunoadhesins, fusion proteins, oligopeptides and other molecules that decrease, block, inhibit, abrogate or interfere with signal transduction resulting from the interaction of PD-L1 with one or more of its binding partners, such as PD-1, B7-1. In some embodiments, a PD-L1 binding agent reduces the negative co-stimulatory signal mediated by or through cell surface proteins expressed on T lymphocytes mediated signaling through PD-L1 so as to render a dysfunctional T cell less dysfunctional (e.g., enhancing effector responses to antigen recognition). In some embodiments, a PD-L1 binding agent is an anti-PD-L1 antibody. In a specific embodiment, an anti-PD-L1 antibody is atezolizumab. In a specific embodiment, an anti-PD-L1 antibody is avelumab. In a specific embodiment, an anti-PD-L1 antibody is durvalumab. In another specific embodiment, an anti-PD-L1 antibody is MDX-1105 (BMS). In another specific embodiment, an anti PD-L1 antibody is MSB0015718C.

The term “PD-L2 binding agent” refers to a molecule that binds PD-L2 and decreases, blocks, inhibits, abrogates or interferes with signal transduction resulting from the interaction of PD-L2 with one or more of its binding partners, such as PD-1. In some embodiments, a PD-L2 binding agent is a molecule that inhibits the binding of PD-L2 to one or more of its binding partners. In some embodiments, the PD-L2 binding agent inhibits binding of PD-L2 to PD-1. In some embodiments, the PD-L2 agents include anti-PD-L2 antibodies, antigen binding fragments thereof, immunoadhesins, fusion proteins, oligopeptides and other molecules that decrease, block, inhibit, abrogate or interfere with signal transduction resulting from the interaction of PD-L2 with one or more of its binding partners, such as PD-1. In some embodiments, a PD-L2 binding agent reduces the negative co-stimulatory signal mediated by or through cell surface proteins expressed on T lymphocytes mediated signaling through PD-L2 so as render a dysfunctional T cell less dysfunctional (e.g., enhancing effector responses to antigen recognition). In some embodiments, a PD-L2 binding agent is an immunoadhesin.

“Pharmaceutical Formulation” refers to a preparation which is in such form as to permit the biological activity of an active ingredient contained therein to be effective, and which contains no additional components which are unacceptably toxic to a subject to which the formulation would be administered. For clarity, DMSO at quantities used in a NeoTCR Product are not considered unacceptably toxic.

A “subject,” “patient,” or an “individual” for purposes of treatment refers to any animal classified as a mammal, including humans, domestic and farm animals, and zoo, sports, or pet animals, such as dogs, horses, cats, cows, etc. Preferably, the mammal is human.

“TCR” as used herein means T cell receptor.

“TIM3 Binding Agent” as used herein means a molecule that binds TIM3 and blocks the binding of TIM3 to galectin-9, phosphatidylserine, HMGB1, and CEACAM1 or another protein that binds to TIM-3.

“Treat,” “Treatment,” and “treating” are used interchangeably and as used herein mean obtaining beneficial or desired results including clinical results. Desirable effects of treatment include, but are not limited to, preventing occurrence or recurrence of disease, alleviation of symptoms, diminishment of any direct or indirect pathological consequences of the disease, preventing metastasis, decreasing the rate of disease progression, amelioration or palliation of the disease state, and remission or improved prognosis. In some embodiments, the NeoTCR Product of the invention are used to delay development of a proliferative disorder (e.g., cancer) or to slow the progression of such disease.

“Tumor antigen” as used herein refers to an antigen (e.g., a polypeptide) that is uniquely or differentially expressed on a tumor cell compared to a normal or non-neoplastic cell. In certain embodiments, a tumor antigen includes any polypeptide expressed by a tumor that is capable of activating or inducing an immune response via an antigen-recognizing receptor or capable of suppressing an immune response via receptor-ligand binding.

“2A” and “2A peptide” are used interchangeably herein and mean a class of 18-22 amino acid long, viral, self-cleaving peptides that are able to mediate cleavage of peptides during translation in eukaryotic cells.

Four well-known members of the 2A peptide class are T2A, P2A, E2A, and F2A. The T2A peptide was first identified in the Thosea asigna virus 2A. The P2A peptide was first identified in the porcine teschovirus-1 2A. The E2A peptide was first identified in the equine rhinitis A virus. The F2A peptide was first identified in the foot-and-mouth disease virus.

The self-cleaving mechanism of the 2A peptides is a result of ribosome skipping the formation of a glycyl-prolyl peptide bond at the C-terminus of the 2A. Specifically, the 2A peptides have a C-terminal conserved sequence that is necessary for the creation of steric hindrance and ribosome skipping. The ribosome skipping can result in one of three options: 1) successful skipping and recommencement of translation resulting in two cleaved proteins (the upstream of the 2A protein which is attached to the complete 2A peptide except for the C-terminal proline and the downstream of the 2A protein which is attached to one proline at the N-terminal; 2) successful skipping but ribosome fall-off that results in discontinued translation and only the protein upstream of the 2A; or 3) unsuccessful skipping and continued translation (i.e., a fusion protein).

### NeoTCR Products.

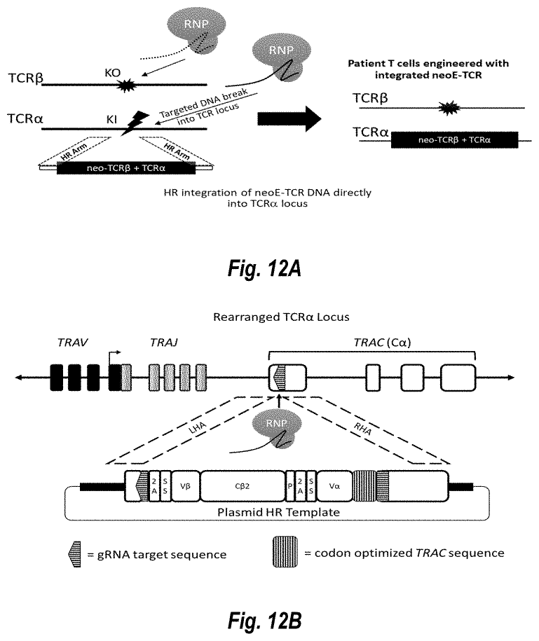

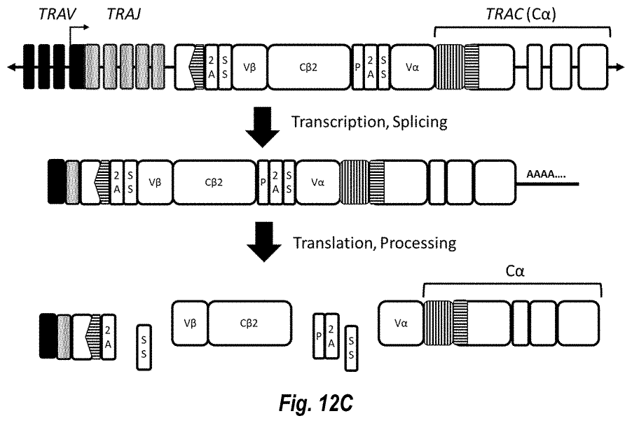

In some embodiments, using the gene editing technology and NeoTCR isolation technology described in PCT/US2020/17887 and PCT/US2019/025415, which are incorporated herein in their entireties, NeoTCRs are cloned in autologous CD8+ and CD4+ T cells from the same patient with cancer by precision genome engineered (using a DNA-mediated (non-viral) method as described in FIGS. 12A-12C) to express the NeoTCR. In other words, the NeoTCRs that are tumor specific are identified in cancer patients, such NeoTCRs are then cloned, and then the cloned NeoTCRs are inserted into the cancer patient's T cells. NeoTCR expressing T cells are then expanded in a manner that preserves a “young” T cell phenotypes, resulting in a NeoTCR-P1 product (i.e., a NeoTCR Product) in which the majority of the T cells exhibit T memory stem cell and T central memory phenotypes. In some embodiments, for example, as a result of the precision genome engineering provided herein, the NeoTCR Product does not comprise any exogenous DNA sequences, e.g., in the genome of the T cells.

These ‘young’ or ‘younger’ or less-differentiated T cell phenotypes are described to confer improved engraftment potential and prolonged persistence post-infusion. Thus, the administration of NeoTCR Product, consisting significantly of ‘young’ T cell phenotypes, has the potential to benefit patients with cancer, through improved engraftment potential, prolonged persistence post-infusion, and rapid differentiation into effector T cells to eradicate tumor cells throughout the body.

Ex vivo mechanism-of-action studies were also performed with NeoTCR Product manufactured with T cells from patients with cancer. Comparable gene editing efficiencies and functional activities, as measured by antigen-specificity of T cell killing activity, proliferation, and cytokine production, were observed demonstrating that the manufacturing process described herein is successful in generating products with T cells from patients with cancer as starting material.

In certain embodiments, the NeoTCR Product manufacturing process involves electroporation of dual ribonucleoprotein species of CRISPR-Cas9 nucleases bound to guide RNA sequences, with each species targeting the genomic TCRα and the genomic TCRβ loci. The specificity of targeting Cas9 nucleases to each genomic locus has been previously described in the literature as being highly specific. Comprehensive testing of the NeoTCR Product was performed in vitro and in silico analyses to survey possible off-target genomic cleavage sites, using COSMID and GUIDE-seq, respectively. Multiple NeoTCR Product or comparable cell products from healthy donors were assessed for cleavage of the candidate off-target sites by deep sequencing, supporting the published evidence that the selected nucleases are highly specific.

Further aspects of the precision genome engineering process have been assessed for safety. No evidence of genomic instability following precision genome engineering was found in assessing multiple NeoTCR Products by targeted locus amplification (TLA) or standard FISH cytogenetics. No off-target integration anywhere into the genome of the NeoTCR sequence was detected. No evidence of residual Cas9 was found in the cell product.

The comprehensive assessment of the NeoTCR Product and precision genome engineering process indicates that the NeoTCR Product will be well tolerated following infusion back to the patient.

The genome engineering approach described herein enables highly efficient generation of bespoke NeoTCR T cells (i.e., NeoTCR Products) for personalized adoptive cell therapy for patients with solid and liquid tumors. Furthermore, the engineering method is not restricted to the use in T cells and has also been applied successfully to other primary cell types, including natural killer and hematopoietic stem cells.

There is increasing evidence that suggests that checkpoint inhibitor-responsive solid tumors are more likely to harbor a higher somatic mutational burden (resulting in expression of tumor-exclusive neoantigens), and the tumors exhibit higher CD8 T cell infiltration and/or exhibit pre-existing high PD-L1 tumor expression (Schumacher & Schreiber, 2015). Each of these features represents a higher potential for endogenous immunogenicity of these tumors, namely that the immune system in those patients will have likely initiated a significant T cell immune response prior to initiation of checkpoint inhibitor therapy (Lawrence, et al, 2013); (Tumeh, et al, 2014); (Wargo, et al, 2017). The application of next generation deep sequencing of tumors and immunologic analysis of the endogenous tumor-targeted T cell response provided compelling evidence for the connection between cancer immunotherapy benefit, tumor mutational burden, and a pre-existing population of neoantigen-specific T cells. The neoantigen-specific population of T cells that specifically recognize and kill the tumor cells harboring these tumor-exclusive mutations (neoantigens) are proposed to be the main mediators of effective cancer immunotherapies to trigger clinical benefit (Tran, et al, 2017) (Schumacher & Schreiber, 2015).

Adoptive TCR-T cell therapy targeting neoepitopes holds the potential to overcome the limitations described above. The NeoTCR Product is a novel adoptive TCR-T cell therapy engineered with autologous neoTCRs of native sequence, identified and isolated from the patient's personal intrinsic T cell cancer immune response. Tumor-specific genomic alterations that initially represent founder (truncal) mutations in each patient, including ‘driver’ mutations for cancer pathology, expand in number and diversify over time as ‘branch’ or ‘passenger’ mutations in later stage malignancies. The spectrum of these accumulated tumor-specific mutations represents a unique private signature of targets for immune recognition in each cancer patient (private neoantigens). T cells that target these private and tumor-exclusive neoantigens (neoepitope or neoE-specific T cells) harbor the potential to exclusively target and kill the tumor cells, while ignoring healthy cells that do not express these tumor-specific mutations. In this way, the immune system of each patient engages the tumors and an appropriately scaled intrinsic immune response, when properly leveraged, has been shown to eradicate the tumors.

Since all cancers are driven by underlying founder or truncal mutations, the NeoTCR Product that targets truncal neoepitopes holds the potential for treatment of any patient with cancer. The NeoTCR Product adoptive personalized cell therapy involves engineering an individual's own CD8 and CD4 T cells to express naturally occurring neoTCRs that already recognize tumor-exclusive neoantigens (neoEs). These neoTCRs, therefore, are of native sequence, derived from pre-existing mutation-targeted CD8 T cells and are captured from peripheral blood by a proprietary isolation technology, which authenticates the tumor-exclusive neoE targets in each patient. In the manufacturing process, freshly derived CD4 and CD8 T cells from a leukopak of the same patient are precision genome engineered to express one neoTCR in a manner that reconstitutes ‘native’ autologous T cell function and that has been validated to interact with the autologous patient predicted antigens throughout the selection process. The clinical benefit to participants with cancer thus stems from delivering a single dose of ex vivo engineered, tumor mutation-targeted autologous NeoTCR cells, thus providing the potential to trigger rapid and durable responses in patients, some of which have no curative treatment options.

The pharmacological evaluation of the NeoTCR Product demonstrated that NeoTCR cells produced with the ex vivo manufacturing process described herein have potent antigen-specific killing, effector cytokine secretion, and proliferative activity on contact with cognate neoantigen-expressing tumor cells. Furthermore, the NeoTCR Product has been shown to respond to target tumor cells with a strong polyfunctional effector protein secretion response, as demonstrated by bulk T cell and single-cell secretome analysis. The observed polyfunctional T cell effector phenotype is predicted to contribute to the potential for clinical benefit upon infusion of NeoTCR Product into patients with cancer in a manner similar to that observed with polyfunctional CAR-T cells infused into patients with hematologic malignancies.

The NeoTCR Product comprise memory stem cell (T_{MSC}) and central memory (T_{CM}) T cell phenotypes as a result of the ex vivo manufacturing process described herein. These ‘younger’ or less-differentiated T cell phenotypes are described to confer improved engraftment potential and prolonged persistence post-infusion in mouse models and in clinical trials of engineered CAR-T cells in patients with hematologic malignancies. Thus, the administration of NeoTCR Product, consisting significantly of ‘younger’ T cell phenotypes has the potential to benefit patients with cancer, through improved engraftment potential, prolonged persistence post-infusion, and rapid differentiation into effector T cells to eradicate tumor cells throughout the body.

Ex vivo mechanism-of-action studies were also performed with NeoTCR Product manufactured with T cells from patients with cancer. Comparable gene editing efficiencies and functional activities, as measured by antigen-specificity of T cell killing activity, proliferation, and cytokine production, were observed demonstrating that the manufacturing process described herein is successful in generating product with T cells from patients with cancer as starting material.

The NeoTCR Product manufacturing process involves electroporation of dual ribonucleoprotein species of CRISPR-Cas9 nucleases bound to guide RNA sequences, with each species targeting the genomic TCRα and the genomic TCRβ loci. The specificity of targeting Cas9 nucleases to each genomic locus has been previously described in the literature as being highly specific. Comprehensive testing of the NeoTCR Product was performed in vitro and in silico analyses to survey possible off-target genomic cleavage sites, using COSMID and GUIDE-seq, respectively. Multiple NeoTCR Product or comparable cell products from healthy donors were assessed for cleavage of the candidate off-target sites by deep sequencing, supporting the published evidence that the selected nucleases are highly specific.

Further aspects of the precision genome engineering process have been assessed for safety. No evidence of genomic instability following precision genome engineering was found in assessing multiple NeoTCR Products by targeted locus amplification (TLA) or standard FISH cytogenetics. No off-target integration anywhere into the genome of the NeoTCR sequence was detected. No evidence of residual Cas9 was found in the cell product.

In certain embodiments, a chemotherapy pre-conditioning regimen may be administered to a Non-Responder Patient prior to the administration of the NeoTCR Product.

Accordingly, the NeoTCR Product provides a novel development in cancer therapy for Non-Responder Patients.

### Checkpoint Inhibitors

Checkpoint inhibitors have been approved over the last several years for the treatment of cancer; however, they are costly, they can result in patient toxicity, and the majority of patients' cancers do not respond to these checkpoint inhibitors. For example, about 20-50% of melanoma and lung cancers will respond significantly to immunotherapies, while others will not.

Furthermore, checkpoint inhibitors that target the PD1, PD-L1, PD-2 and PD-L2 (the “PD-1 Axis” and “PD-1 Axis Binding Agents”) have been widely explored and, like other checkpoint inhibitors, suffer from toxicity and lack of efficacy in the majority of cancers (see Table 1 for a summary of select checkpoint inhibitor breast cancer clinical trials). Even though diagnostics can be used to help predict response rates (e.g., PD-L1 expression by tumor cells prior to treatment has been used to try to predict response rates to anti-PD-1 and anti-PD-L1 therapy), the majority of patients with PD-L1(+) tumors do not respond to PD-1 pathway blockade and there is no previously disclosed diagnostic that accurately predicts response rate.

TABLE 1
Low Efficacy and Adverse Events of Exemplary Checkpoint Inhibitors

| | Study | % Patients | % Patients | | 1-yr OS | |
|---|---|---|---|---|---|---|
| Cancer Type | Description | with AEs | Grade 3-4 AEs | ORR (%) | rate (%) | Reference |
| Metastatic/ | Atezolizumab, | 66 | 16 | 23 | 57 | Balar et al. |
| Advanced | PhII, 1,200 mg | | | | | 2017; |
| Breast Cancer | IV q3wk | | | | | The Lancet |
| Metastatic/ | Atezolizumab, | 69 | 16 | 15 | 37 | Rosenbert et al. |
| Advanced | PhII, 1,200 mg | | | | | 2016; |
| Breast Cancer | IV q3wk | | | | | The Lancet |
| Metastatic | Atezolizumab, | 57.4 | 4.40 | NR | NR | Chen et al. |
| Breast Cancer | PhI, 1,200 mg | | | | | 2014; |
| | IV q3wk | | | | | Nature |
| Metastatic/ | Atezolizumab, | 70 | 16 | 16 | NR | Loriot et al. |
| Advanced | PhII, 1,200 mg | | | | | 2016; |
| Breast Cancer | IV q3wk | | | | | Annals of Onc |
| Metastatic | Atezolizumab, | 57 | 4 | 26 | NR | Powles et al. |
| Breast Cancer | PhII, 15 mg/kg | | | | | 2014, |
| | IV q3wk | | | | | Annals of Oncology |
| Metastatic/ | Durvalumab, | 60.70 | 6.80 | 17.80 | 55 | Powles et al. |
| Advanced | PhI/II, 10 mg/kg | | | | | 2017; |
| Breast Cancer | IV q2wk | | | | | JAMA Onc |
| Advanced | Durvalumab, | 63.90 | 4.90 | 31 | NR | Massard et al. |
| Breast Cancer | PhI/II, 10 mg/kg | | | | | 2016; J of |
| | IV q2wk | | | | | Clinical Oncology |
| Metastatic/ | Durvalumab, | 50 | 0 | 60 | 44 | Levey et al. |
| Advanced | PhI/II, 10 mg/kg | | | | | 2016; European |
| Breast Cancer | IV q2wk | | | | | J of Cancer |
| Metastatic | Nivolumab, | 59 | 22 | 24.4 | 46 | Sharma et al. |
| Breast Cancer | PhI/II, 3 mg/kg | | | | | 2016; |
| | IV q2wk | | | | | The Lancet Onc |
| Metastatic | Nivolumab, | 64 | 18 | 19.60 | NR | Sharma et al. |
| Breast Cancer | PhI/II, 3 mg/kg | | | | | 2017; |
| | IV q2wk | | | | | The Lancet Onc |
| Advanced | Pembrolizumab, | 60.90 | 15 | 21.10 | 43.90 | Bellmunt et al. |
| Breast Cancer | PhIII, 200 mg | | | | | 2017; New England |
| | IV q3wk | | | | | J of Med |
| Metastatic/ | Pembrolizumab, | 45 | 15 | 26 | 50 | Plimack et al. |
| Advanced | PhIB, 10 mg/kg | | | | | 2017; |
| Breast Cancer | IV q2wk | | | | | The Lancet Onc. |
| Metastatic/ | Pembrolizumab, | 62 | 16 | 24 | NR | Balar et al |
| Advanced | PhII, 200 mg | | | | | 2017; |
| Breast Cancer | IV q3wk | | | | | The Lancet Onc |
| Metastatic | Avelumab, | 65.9 | 6.80 | 18.2 | 54.30 | Apolo et al. |
| Breast Cancer | PhIB, 10 mg/kg | | | | | 2017; |
| | IV q2wk | | | | | J Clin Onc |

Adapted from Fan et al. Onco Targets Ther. 2019; 12: 1791-1801.

In addition to PD-1 Axis Binding Agents, other checkpoint inhibitors exhibit the same limitations—primarily, toxicity and lack of efficacy (non-response and acquired resistance). Checkpoint inhibitors include but are not limited to PD-1 Binding Agents, PD-L1 Binding Agents, PD-L2 Binding Agents, CTLA-4 Binding Agents, T-cell immunoglobulin and mucin domain-3 (TIM3) Binding Agents, T lymphocyte markers (including but not limited to lymphocyte activation gene 3 (LAG-3), TIM-3 (as described herein), V-domain containing Ig Suppressor of T cell Activation (VISTA), T cell immunoglobulin and ITIM domain (TIGIT), B7-H3, inducible T-cell co-stimulator (ICOS/ICOS-L), CD27/CD70), and glucocorticoid-induced TNF Receptor (GITR), macrophage markers (including but not limited to CD47/signal regulatory protein alpha (SIRPα) and indoleamine-2,3-dioxygenase (IDO)), natural killer cell markers (including but not limited to CD94/NKG2A and the killer immunoglobulin-like receptor family (KIR), and other agents that block proteins that stop the immune system from killing and/or stopping or slowing the proliferation of cancer cells.

Certain PD-1 Axis Binding Agents include but are not limited to pembrolizumab, nivolumab, cemiplimab, and other monoclonal antibodies that bind to PD-1. Certain PD-L1 Axis Binding Agents include but are not limited to atezolizumab, avelumab, durvalumab, and other monoclonal antibodies that bind to PD-L1. As discussed herein, one limitation of these agents is that have been shown to allow the immune system to attack some normal cells and organs in a patient which leads to serious side effects. Additional side effects of these agents include but are not limited to serious problems in the lungs, intestines, liver, kidneys, hormone-making glands, or other organs.

Certain CTLA-4 Binding Agents include ipilimumab and other monoclonal antibodies that bind to CTLA-4. Similar to the PD-1 Axis Binding Agents, CTLA-4 Binding Agents cause serious and life-threatening side effects and they are not effective for treating all cancers and all patients having cancer.

Certain TIM-3 Binding Agents include monoclonal antibodies that bind to TIM-3. Similar to the PD-1 Axis Binding Agents, TIM-3 Binding Agents can cause serious and life-threatening side effects and they are not effective for treating all cancers and all patients having cancer.

It is clear that there is a need for a therapy to treat cancers (and patients who have cancer) that do not respond to checkpoint inhibitors either because they don't respond to the treatment (either no response or partial response) or they build up an acquired resistance to the treatment (i.e., collectively, Non-Responder Patients). Furthermore, there is also a need for a therapy to augment and improve the response (e.g., overall response rates (ORR) and overall survival (OS) rates).

Accordingly, the NeoTCR Product provides a novel development in cancer therapy for Non-Responder Patients.

### Cancer Immunotherapies

Cancer immunotherapies, i.e., the broader class of therapies to which checkpoint inhibitors are classified, have been approved over the last several years for the treatment of cancer; however, they are costly, they can result in patient toxicity, and the majority of patients' cancers do not respond to these checkpoint inhibitors. For example, cancer immunotherapies include but are not limited to the checkpoint inhibitors described herein, CAR-T therapies, cancer vaccines, and cytokine therapies (i.e., cytokines and modifications, derivatives, and fusion proteins thereof that are formulated into a pharmaceutical formulation). Approximately 60-70% of tumors are not responsive to single-agent checkpoint inhibitors and patients who do have tumors that respond to checkpoint inhibitors become resistant over time (Yan et al., Front Immunol. 208; 9:1739).

Based on the low response rate of cancer immunotherapies and the high rate of resistance, it is clear that there is a need for a therapy to treat cancers (and patients who have cancer) that do not respond to immunotherapies either because they don't respond to the treatment (no response or only a partial response) or because they build up an acquired resistance to the treatment (i.e., collectively, Non-Responder Patients). Furthermore, there is also a need for a therapy to augment and improve the response (e.g., overall response rates (ORR) and overall survival (OS) rates).

Accordingly, the NeoTCR Product provides a novel development in cancer therapy for Non-Responder Patients.

### Gene-Editing Methods

In certain embodiments, the present disclosure involves, in part, methods of engineering human cells, e.g., engineered T cells or engineered human stem cells. In certain embodiments, the present disclosure involves, in part, methods of engineering human cells, e.g., NK cells, NKT cells, macrophages, hematopoietic stem cells (HSCs), cells derived from HSCs, or dendritic/antigen-presenting cells. In certain embodiments, such engineering involves genome editing. For example, but not by way of limitation, such genome editing can be accomplished with nucleases targeting one or more endogenous loci, e.g., TCR alpha (TCRα) locus and TCR beta (TCRβ) locus. In certain embodiments, the nucleases can generate single-stranded DNA nicks or double-stranded DNA breaks in an endogenous target sequence. In certain embodiments, the nuclease can target coding or non-coding portions of the genome, e.g., exons, introns. In certain embodiments, the nucleases contemplated herein comprise homing endonuclease, meganuclease, megaTAL nuclease, transcription activator-like effector nuclease (TALEN), zinc-finger nuclease (ZFN), and clustered regularly interspaced short palindromic repeats (CRISPR)/Cas nuclease. In certain embodiments, the nucleases can themselves be engineered, e.g., via the introduction of amino acid substitutions and/or deletions, to increase the efficiency of the cutting activity.

In certain embodiments, a CRISPR/Cas nuclease system is used to engineer human cells. In certain embodiments, the CRISPR/Cas nuclease system comprises a Cas nuclease and one or more RNAs that recruit the Cas nuclease to the endogenous target sequence, e.g., single guide RNA. In certain embodiments, the Cas nuclease and the RNA are introduced in the cell separately, e.g. using different vectors or compositions, or together, e.g., in a polycistronic construct or a single protein-RNA complex. In certain embodiments, the Cas nuclease is Cas9 or Cas12a. In certain embodiments, the Cas9 polypeptide is obtained from a bacterial species including, without limitation, Streptococcus pyogenes or Neisseria menengitidis. Additional examples of CRISPR/Cas systems are known in the art. See Adli, Mazhar. “The CRISPR tool kit for genome editing and beyond.” Nature communications vol. 9,1 1911 (2018), herein incorporated by reference for all that it teaches.

In certain embodiments, genome editing occurs at one or more genome loci that regulate immunological responses. In certain embodiments, the loci include, without limitation, TCR alpha (TCRα) locus, TCR beta (TCRβ) locus, TCR gamma (TCRγ), and TCR delta (TCRδ).

In certain embodiments, genome editing is performed by using non-viral delivery systems. For example, a nucleic acid molecule can be introduced into a cell by administering the nucleic acid in the presence of lipofection (Feigner et al., Proc. Natl. Acad. Sci. U.S.A. 84:7413, 1987; Ono et al., Neuroscience Letters 17:259, 1990; Brigham et al., Am. J. Med. Sci. 298:278, 1989; Staubinger et al., Methods in Enzymology 101:512, 1983), asialoorosomucoid-polylysine conjugation (Wu et al., Journal of Biological Chemistry 263:14621, 1988; Wu et al., Journal of Biological Chemistry 264:16985, 1989), or by micro-injection under surgical conditions (Wolff et al., Science 247:1465, 1990). Other non-viral means for gene transfer include transfection in vitro using calcium phosphate, DEAE dextran, electroporation, and protoplast fusion. Liposomes can also be potentially beneficial for delivery of DNA into a cell. Transplantation of normal genes into the affected tissues of a subject can also be accomplished by transferring a normal nucleic acid into a cultivatable cell type ex vivo (e.g., an autologous or heterologous primary cell or progeny thereof), after which the cell (or its descendants) are injected into a targeted tissue or are injected systemically.

In certain embodiments, genome editing is performed by using viral delivery systems. In certain embodiments, the viral methods include targeted integration (including but not limited to AAV) and random integration (including but not limited to lentiviral approaches). In certain embodiments, the viral delivery would be accomplished without integration of the nuclease. In such embodiments, the viral delivery system can be Lentiflash or another similar delivery system.

### Homology Recombination Templates

In certain embodiments, the present disclosure provides genome editing of a cell by introducing and recombining a homologous recombination (HR) template nucleic acid sequence into an endogenous locus of a cell. In certain embodiments, the HR template nucleic acid sequence is linear. In certain embodiments, the HR template nucleic acid sequence is circular. In certain embodiments, the circular HR template can be a plasmid, minicircle, or nanoplasmid. In certain embodiments, the HR template nucleic acid sequence comprises a first and a second homology arms. In certain embodiments, the homology arms can be of about 300 bases to about 2,000 bases. For example, each homology arm can be 1,000 bases. In certain embodiments, the homology arms can be homologous to a first and second endogenous sequences of the cell. In certain embodiments, the endogenous locus is a TCR locus. For example, the first and second endogenous sequences are within a TCR alpha locus or a TCR beta locus. In certain embodiments, the HR template comprises a TCR gene sequences. In non-limiting embodiments, the TCR gene sequence is a patient specific TCR gene sequence. In non-limiting embodiments, the TCR gene sequence is tumor-specific. In non-limiting embodiments, the TCR gene sequence can be identified and obtained using the methods described in PCT/US2020/017887, the content of which is herein incorporated by reference. In certain embodiments, the HR template comprises a TCR alpha gene sequence and a TCR beta gene sequence.

In certain embodiments, the HR template is a polycistronic polynucleotide. In certain embodiments, the HR template comprises sequences encoding for flexible polypeptide sequences (e.g., Gly-Ser-Gly sequence). In certain embodiments, the HR template comprises sequences encoding an internal ribosome entry site (IRES). In certain embodiments, the HR template comprises a 2A peptide (e.g., P2A, T2A, E2A, and F2A). Additional information on the HR template nucleic acids and methods of modifying a cell thereof can be found in International Patent Application no. PCT/US2018/058230, the content of which is herein incorporated by reference.

### Methods of Treatment

The NeoTCR Products disclosed herein can be used to treat Non-Responder Patients.

In certain embodiments, the NeoTCR Products can be administered to a Non-Responder Patient after such patient has previously been treated with a checkpoint inhibitor.

In certain embodiments, the NeoTCR Products can be administered to a checkpoint inhibitor naïve Non-Responder Patient.

In certain embodiments, the NeoTCR Products can be administered to a Non-Responder Patient concurrently with a checkpoint inhibitor. In certain embodiments, the NeoTCR Products can be administered to a Non-Responder Patient sequentially with a checkpoint inhibitor. In certain embodiments, the checkpoint inhibitor is an CTLA-4 Binding Agent. In certain embodiments, the checkpoint inhibitor is an anti-CTLA antibody. In certain embodiments, the checkpoint inhibitor is ipilimumab. In certain embodiments, the checkpoint inhibitor is a PD-1 Axis Binding Agent. In certain embodiments, the checkpoint inhibitor is a PD-1 Binding Agent. In certain embodiments, the checkpoint inhibitor is pembrolizumab. In certain embodiments, the checkpoint inhibitor is nivolumab. In certain embodiments, the checkpoint inhibitor is cemiplimab. In certain embodiments, the checkpoint inhibitor is a PD-L1 Binding Agent. In certain embodiments, the checkpoint inhibitor is atezolizumab. In certain embodiments, the checkpoint inhibitor is avelumab. In certain embodiments, the checkpoint inhibitor is durvalumab. In certain embodiments, the checkpoint inhibitor is a PD-L2 Binding Agent. In certain embodiments, the NeoTCR Products can be administered to a Non-Responder Patient in combination with any other chemotherapeutic agent that is standard of care or otherwise an acceptable agent for treating such a patient based on factors such as disease, age, medical history, and other factors that a medical physician would consider.

In certain embodiments, the NeoTCR Products can be administered to a Non-Responder Patient after such patient has previously been treated with an immunotherapy.

In certain embodiments, the NeoTCR Products can be administered to a Non-Responder Patient concurrently with an immunotherapy. In certain embodiments, the NeoTCR Products can be administered to a Non-Responder Patient sequentially with an immunotherapy. In certain embodiments, the immunotherapy is a cancer vaccine. In certain embodiments, the immunotherapy is a cytokine therapies (i.e., cytokines and modifications, derivatives, and fusion proteins thereof that are formulated into a pharmaceutical formulation). In certain embodiments, the immunotherapy is a cell therapy other than a NeoTCR Product. In certain embodiments, the immunotherapy is a CAR-T cell therapy. In certain embodiments, the immunotherapy is an interferon. In certain embodiments, the immunotherapy is an interleukin. In certain embodiments, the immunotherapy is an oncolytic virus therapy. In certain embodiments, the immunotherapy is an NK cell therapy. In certain embodiments, the immunotherapy is a T cell therapy other than NeoTCR Products. In certain embodiments, the NeoTCR Products can be administered to a Non-Responder Patient in combination with any other chemotherapeutic agent that is standard of care or otherwise an acceptable agent for treating such a patient based on factors such as disease, age, medical history, and other factors that a medical physician would consider.

In certain embodiments, an effective amount of the NeoTCR Product is delivered through IV administration. In certain embodiments, the NeoTCR Products are delivered through IV administration in a single administration. In certain embodiments, the NeoTCR Products are delivered through IV administration in multiple administrations. In certain embodiments, the NeoTCR Products are delivered through IV administration in two or more administrations. In certain embodiments, the NeoTCR Products are delivered through IV administration in two administrations. In certain embodiments, the NeoTCR Products are delivered through IV administration in three administrations.

Non-limiting examples of cancer include blood cancers (e.g. leukemias, lymphomas, and myelomas), ovarian cancer, breast cancer, bladder cancer, brain cancer, colon cancer, intestinal cancer, liver cancer, lung cancer, pancreatic cancer, prostate cancer, skin cancer, stomach cancer, glioblastoma, throat cancer, melanoma, neuroblastoma, adenocarcinoma, glioma, soft tissue sarcoma, and various carcinomas (including prostate and small cell lung cancer). Suitable carcinomas further include any known in the field of oncology, including, but not limited to, astrocytoma, fibrosarcoma, myxosarcoma, liposarcoma, oligodendroglioma, ependymoma, medulloblastoma, primitive neural ectodermal tumor (PNET), chondrosarcoma, osteogenic sarcoma, pancreatic ductal adenocarcinoma, small and large cell lung adenocarcinomas, chordoma, angiosarcoma, endotheliosarcoma, squamous cell carcinoma, bronchoalveolarcarcinoma, epithelial adenocarcinoma, and liver metastases thereof, lymphangiosarcoma, lymphangioendotheliosarcoma, hepatoma, cholangiocarcinoma, synovioma, mesothelioma, Ewing's tumor, rhabdomyosarcoma, colon carcinoma, basal cell carcinoma, sweat gland carcinoma, papillary carcinoma, sebaceous gland carcinoma, papillary adenocarcinoma, cystadenocarcinoma, medullary carcinoma, bronchogenic carcinoma, renal cell carcinoma, bile duct carcinoma, choriocarcinoma, seminoma, embryonal carcinoma, Wilms' tumor, testicular tumor, medulloblastoma, craniopharyngioma, ependymoma, pinealoma, hemangioblastoma, acoustic neuroma, oligodendroglioma, meningioma, neuroblastoma, retinoblastoma, leukemia, multiple myeloma, Waldenstrom's macroglobulinemia, and heavy chain disease, breast tumors such as ductal and lobular adenocarcinoma, squamous and adenocarcinomas of the uterine cervix, uterine and ovarian epithelial carcinomas, prostatic adenocarcinomas, transitional squamous cell carcinoma of the bladder, B and T cell lymphomas (nodular and diffuse) plasmacytoma, acute and chronic leukemias, malignant melanoma, soft tissue sarcomas and leiomyosarcomas. In certain embodiments, the neoplasia is selected from blood cancers (e.g. leukemias, lymphomas, and myelomas), ovarian cancer, prostate cancer, breast cancer, bladder cancer, brain cancer, colon cancer, intestinal cancer, liver cancer, lung cancer, pancreatic cancer, prostate cancer, skin cancer, stomach cancer, glioblastoma, and throat cancer. In certain embodiments, the presently disclosed young T cells and compositions comprising thereof can be used for treating and/or preventing blood cancers (e.g., leukemias, lymphomas, and myelomas) or ovarian cancer, which are not amenable to conventional therapeutic interventions.

In certain embodiments, the neoplasia is a solid cancer or a solid tumor. In certain embodiments, the solid tumor or solid cancer is selected from glioblastoma, prostate adenocarcinoma, kidney papillary cell carcinoma, sarcoma, ovarian cancer, pancreatic adenocarcinoma, rectum adenocarcinoma, colon adenocarcinoma, esophageal carcinoma, uterine corpus endometrioid carcinoma, breast cancer, skin cutaneous melanoma, lung adenocarcinoma, stomach adenocarcinoma, cervical and endocervical cancer, kidney clear cell carcinoma, testicular germ cell tumors, and aggressive B-cell lymphomas.

The subjects can have an advanced form of disease, in which case the treatment objective can include mitigation or reversal of disease progression, and/or amelioration of side effects. The subjects can have a history of the condition, for which they have already been treated, in which case the therapeutic objective will typically include a decrease or delay in the risk of recurrence.

Suitable human subjects for therapy typically comprise two treatment groups that can be distinguished by clinical criteria. Subjects with “advanced disease” or “high tumor burden” are those who bear a clinically measurable tumor. A clinically measurable tumor is one that can be detected on the basis of tumor mass (e.g., by palpation, CAT scan, sonogram, mammogram or X-ray; positive biochemical or histopathologic markers on their own are insufficient to identify this population). A pharmaceutical composition is administered to these subjects to elicit an anti-tumor response, with the objective of palliating their condition. Ideally, reduction in tumor mass occurs as a result, but any clinical improvement constitutes a benefit. Clinical improvement includes decreased risk or rate of progression or reduction in pathological consequences of the tumor.

### Compositions and Articles of Manufacture

The NeoTCR Products can be used in combination with articles of manufacture. Such articles of manufacture can be useful for the prevention or treatment of proliferative disorders (e.g., cancer). Examples of articles of manufacture include but are not limited to containers (e.g., infusion bags, bottles, storage containers, flasks, vials, syringes, tubes, and IV solution bags) and a label or package insert on or associated with the container. The containers may be made of any material that is acceptable for the storage and preservation of the NeoTCR Cells within the NeoTCR Products. In certain embodiments, the container may be an intravenous solution bag or a vial having a stopper pierceable by a hypodermic injection needle. For example, the container may be a CryoMACS freezing bag. The label or package insert indicates that the NeoTCR Products are used for treating the condition of choice and the patient of origin. The patient is identified on the container of the NeoTCR Product because the NeoTCR Products is made from autologous cells and engineered as a patient-specific and individualized treatment.

The article of manufacture may comprise: 1) a first container with a NeoTCR Product contained therein.

The article of manufacture may comprise: 1) a first container with a NeoTCR Product contained therein; and 2) a second container with the same NeoTCR Product as the first container contained therein. Optionally, additional containers with the same NeoTCR Product as the first and second containers may be prepared and made. Optionally, additional containers containing a composition comprising a different cytotoxic or otherwise therapeutic agent may also be combined with the containers described above.

The article of manufacture may comprise: 1) a first container with a NeoTCR Product contained therein; and 2) a second container with a composition contained therein, wherein the composition comprises a further cytotoxic or otherwise therapeutic agent.

The article of manufacture may comprise: 1) a first container with two NeoTCR Products contained therein; and 2) a second container with a composition contained therein, wherein the composition comprises a further cytotoxic or otherwise therapeutic agent.

The article of manufacture may comprise: 1) a first container with a NeoTCR Product contained therein; 2) a second container with a second NeoTCR Product contained therein; and 3) optionally a third container with a composition contained therein, wherein the composition comprises a further cytotoxic or otherwise therapeutic agent. In certain embodiments, the first and second NeoTCR Products are different NeoTCR Products. In certain embodiments, the first and second NeoTCR Products are the same NeoTCR Products.

The article of manufacture may comprise: 1) a first container with three NeoTCR Products contained therein; and 2) optionally a second container with a composition contained therein, wherein the composition comprises a further cytotoxic or otherwise therapeutic agent.

The article of manufacture may comprise: 1) a first container with a NeoTCR Product contained therein; 2) a second container with a second NeoTCR Product contained therein; 3) a third container with a third NeoTCR Product contained therein; and 4) optionally a fourth container with a composition contained therein, wherein the composition comprises a further cytotoxic or otherwise therapeutic agent. In certain embodiments, the first, second, and third NeoTCR Products are different NeoTCR Products. In certain embodiments, the first, second, and third NeoTCR Products are the same NeoTCR Products. In certain embodiments, two of the first, second, and third NeoTCR Products are the same NeoTCR Products.

The article of manufacture may comprise: 1) a first container with four NeoTCR Products contained therein; and 2) optionally a second container with a composition contained therein, wherein the composition comprises a further cytotoxic or otherwise therapeutic agent.

The article of manufacture may comprise: 1) a first container with a NeoTCR Product contained therein; 2) a second container with a second NeoTCR Product contained therein; 3) a third container with a third NeoTCR Product contained therein; 4) a fourth container with a fourth NeoTCR Product contained therein; and 5) optionally a fifth container with a composition contained therein, wherein the composition comprises a further cytotoxic or otherwise therapeutic agent. In certain embodiments, the first, second, third, and fourth NeoTCR Products are different NeoTCR Products. In certain embodiments, the first, second, third, and fourth NeoTCR Products are the same NeoTCR Products. In certain embodiments, two of the first, second, third, and fourth NeoTCR Products are the same NeoTCR Products. In certain embodiments, three of the first, second, third, and fourth NeoTCR Products are the same NeoTCR Products.

The article of manufacture may comprise: 1) a first container with five or more NeoTCR Products contained therein; and 2) optionally a second container with a composition contained therein, wherein the composition comprises a further cytotoxic or otherwise therapeutic agent.

The article of manufacture may comprise: 1) a first container with a NeoTCR Product contained therein; 2) a second container with a second NeoTCR Product contained therein; 3) a third container with a third NeoTCR Product contained therein; 4) a fourth container with a fourth NeoTCR Product contained therein; 5) a fifth container with a fifth NeoTCR Product contained therein; 6) optionally a sixth or more additional containers with a sixth or more NeoTCR Product contained therein; and 7) optionally an additional container with a composition contained therein, wherein the composition comprises a further cytotoxic or otherwise therapeutic agent. In certain embodiments, all of the containers of NeoTCR Products are different NeoTCR Products. In certain embodiments, all of the containers of NeoTCR Products are the same NeoTCR Products. In certain embodiments, there can be any combination of same or different NeoTCR Products in the five or more containers based on the availability of detectable NeoTCRs in a patient's tumor sample(s), the need and/or desire to have multiple NeoTCR Products for the patient, and the availability of any one NeoTCR Product that may require or benefit from one or more container.

The article of manufacture may comprise: 1) a first container with a NeoTCR Product contained therein; 2) a second container with a second NeoTCR Product contained therein; and 3) a third container with a third NeoTCR Product contained therein.

The article of manufacture may comprise: 1) a first container with a NeoTCR Product contained therein; 2) a second container with a second NeoTCR Product contained therein; 3) a third container with a third NeoTCR Product contained therein; and 4) optionally a fourth container with a fourth NeoTCR Product contained therein.

The article of manufacture may comprise: 1) a first container with a NeoTCR Product contained therein; 2) a second container with a second NeoTCR Product contained therein; 3) a third container with a third NeoTCR Product contained therein; 4) a fourth container with a fourth NeoTCR Product contained therein; and 5) optionally a fifth container with a fourth NeoTCR Product contained therein.

The article of manufacture may comprise a container with one NeoTCR Product contained therein. The article of manufacture may comprise a container with two NeoTCR Products contained therein. The article of manufacture may comprise a container with three NeoTCR Products contained therein. The article of manufacture may comprise a container with four NeoTCR Products contained therein. The article of manufacture may comprise a container with five NeoTCR Products contained therein.

The article of manufacture may comprise 1) a first container with one NeoTCR Product contained therein, and 2) a second container with two NeoTCR Products contained therein. The article of manufacture may comprise 1) a first container with two NeoTCR Products contained therein, and 2) a second container with one NeoTCR Product contained therein. In the examples above, a third and/or fourth container comprising one or more additional NeoTCR Products may be included in the article of manufacture. Additionally, a fifth container comprising one or more additional NeoTCR Products may be included in the article of manufacture.

Furthermore, any container of NeoTCR Product described herein can be split into two, three, or four separate containers for multiple time points of administration and/or based on the appropriate dose for the patient.

In certain embodiments, the NeoTCR Products are provided in a kit. The kit can, by means of non-limiting examples, contain package insert(s), labels, instructions for using the NeoTCR Product(s), syringes, disposal instructions, administration instructions, tubing, needles, and anything else a clinician would need in order to properly administer the NeoTCR Product(s).

The NeoTCR Products disclosed herein that are used to treat Non-Responder Patients are formulated into pharmaceutical formulation for the use thereof. In certain embodiments, the label of such NeoTCR Products provides instructions for treating Non-Responder Patients.

In certain embodiments, the label of such NeoTCR Products provides instructions for treating Non-Responder Patients who have previously undergone treatment with a checkpoint inhibitor. In certain embodiments, the label of such NeoTCR Products provides instructions for treating Non-Responder Patients who are checkpoint inhibitor naïve. In certain embodiments, the label of such NeoTCR Products provides instructions for treating Non-Responder Patients who will concurrently undergo treatment with a checkpoint inhibitor. In certain embodiments, the label of such NeoTCR Products provides instructions for treating Non-Responder Patients who will sequentially undergo treatment with a checkpoint inhibitor.

In certain embodiments, the label of such NeoTCR Products provides instructions for treating Non-Responder Patients who have previously undergone treatment with an immunotherapy. In certain embodiments, the label of such NeoTCR Products provides instructions for treating Non-Responder Patients who are immunotherapy naïve. In certain embodiments, the label of such NeoTCR Products provides instructions for treating Non-Responder Patients who will concurrently undergo treatment with an immunotherapy. In certain embodiments, the label of such NeoTCR Products provides instructions for treating Non-Responder Patients who will sequentially undergo treatment with an immunotherapy.

Also provided is a method of treating cancer in a patient. In certain embodiments the method comprises identifying the patient as a Non-Responder to a checkpoint inhibitor in accordance with the methods known in the art and treating the patient with a NeoTCR Product. In certain embodiments the method comprises identifying the patient as a Non-Responder to a checkpoint inhibitor in accordance with the methods known in the art and treating the patient with a NeoTCR Product if the patient is checkpoint inhibitor naïve. In certain embodiments the method comprises identifying the patient as a Non-Responder to a checkpoint inhibitor in accordance with the methods known in the art and treating the patient with a NeoTCR Product if the patient has previously received a checkpoint inhibitor. In certain embodiments the method comprises identifying the patient as a Non-Responder to a checkpoint inhibitor in accordance with the methods known in the art and treating the patient with a NeoTCR Product if the patient is currently undergoing therapy with a checkpoint inhibitor. In certain embodiments, the checkpoint inhibitor is an CTLA-4 Binding Agent. In certain embodiments, the checkpoint inhibitor is an anti-CTLA antibody. In certain embodiments, the checkpoint inhibitor is ipilimumab. In certain embodiments, the checkpoint inhibitor is a PD-1 Axis Binding Agent. In certain embodiments, the checkpoint inhibitor is a PD-1 Binding Agent. In certain embodiments, the checkpoint inhibitor is pembrolizumab. In certain embodiments, the checkpoint inhibitor is nivolumab. In certain embodiments, the checkpoint inhibitor is cemiplimab. In certain embodiments, the checkpoint inhibitor is a PD-L1. Binding Agent. In certain embodiments, the checkpoint inhibitor is atezolizumab. In certain embodiments, the checkpoint inhibitor is avelumab. In certain embodiments, the checkpoint inhibitor is durvalumab. In certain embodiments, the checkpoint inhibitor is a PD-L2 Binding Agent.

Also provided is a method of treating cancer in a patient with a NeoTCR Product and optionally an immunotherapy. In certain embodiments the method comprises identifying the patient as a Non-Responder to an immunotherapy in accordance with the methods known in the art and treating the patient with a NeoTCR Product. In certain embodiments the method comprises identifying the patient as a Non-Responder to an immunotherapy in accordance with the methods known in the art and treating the patient with a NeoTCR Product if the patient is immunotherapy naïve. in certain embodiments the method comprises identifying the patient as a Non-Responder to an immunotherapy in accordance with the methods known in the art and treating the patient with a NeoTCR Product if the patient has previously received an immunotherapy. In certain embodiments the method comprises identifying the patient as a Non-Responder to an immunotherapy in accordance with the methods known in the art and treating the patient with a NeoTCR Product if the patient is currently undergoing therapy with an immunotherapy. In certain embodiments, the immunotherapy is a cancer vaccine. In certain embodiments, the immunotherapy is a cytokine therapies (i.e., cytokines and modifications, derivatives, and fusion proteins thereof that are formulated into a pharmaceutical formulation). In certain embodiments, the immunotherapy is a cell therapy other than a NeoTCR Product. In certain embodiments, the immunotherapy is a CAR-T therapy. In certain embodiments, the immunotherapy is an interferon. In certain embodiments, the immunotherapy is an interleukin. In certain embodiments, the immunotherapy is an oncolytic virus therapy. In certain embodiments, the immunotherapy is an NK cell therapy. In certain embodiments, the immunotherapy is a T cell therapy other than NeoTCR Products. In certain embodiments, the NeoTCR Products can be administered to a Non-Responder Patient in combination with any other chemotherapeutic agent that is standard of care or otherwise an acceptable agent for treating such a patient based on factors such as disease, age, medical history, and other factors that a medical physician would consider.

In certain embodiments, the NeoTCR Products are provided in a kit with instructions for treating Non-Responder Patients who did not respond to an immunotherapy.

### Therapeutic Compositions And Methods Of Manufacturing

As described herein, plasmid DNA-mediated precision genome engineering process for Good Manufacturing Practice (GMP) manufacturing of NeoTCR Products was developed. Targeted integration of the patient-specific neoTCR was accomplished by electroporating CRISPR endonuclease ribonucleoproteins (RNPs) together with the personalized neoTCR gene cassette, encoded by the plasmid DNA. In addition to the neoTCR, the NeoTCR Constructs were inserted by incorporating them into the neoTCR vector and then electroporating with CRISPR endonuclease ribonucleoproteins (RNPs) as described above.

The NeoTCR Products can be formulated into a drug product using the clinical manufacturing process. Under this process, the NeoTCR Products are cryopreserved in CryoMACS Freezing Bags. One or more bags may be shipped to the site for each patient depending on patient needs. The product is composed of apheresis-derived, patient-autologous, CD8 and CD4 T cells that have been precision genome engineered to express one or more autologous neoTCRs targeting a neoepitope complexed to one of the endogenous HLA receptors presented exclusively on the surface of that patient's tumor cells.

The final product will contain 5% dimethyl sulfoxide (DMSO), human serum albumin, and Plasma-Lyte. The final cell product will contain Total nucleated NeoTCR cells (cGMP manufactured), Plasma-Lyte A (USP), HAS in 0.02-0.08 M sodium caprylate and sodium tryptophanate (USP), and CryoStor CS10 (cGMP manufactured with USP grade materials).

### Compositions and Vectors

The presently disclosed subject matter provides compositions comprising cells (e.g., immunoresponsive cells) disclosed herein.

In certain embodiments, the presently disclosed subject matter provides nucleic acid compositions comprising a polynucleotide encoding the NeoTCR disclosed herein. In certain embodiments, the nucleic acid compositions disclosed herein comprise a polynucleotide encoding a NeoTCR Construct disclosed herein. Also provided are cells comprising such nucleic acid compositions.

In certain embodiments, the nucleic acid composition further comprises a promoter that is operably linked to the NeoTCR disclosed herein. In certain embodiments, the nucleic acid composition further comprises a promoter that is operably linked to the NeoTCR Construct disclosed herein.

In certain embodiments, the promoter is endogenous or exogenous. In certain embodiments, the exogenous promoter is selected from an elongation factor (EF)-1 promoter, a CMV promoter, a SV40 promoter, a PGK promoter, a long terminal repeat (LTR) promoter and a metallothionein promoter. In certain embodiments, the promoter is an inducible promoter. In certain embodiments, the inducible promoter is selected from a NFAT transcriptional response element (TRE) promoter, a CD69 promoter, a CD25 promoter, an IL-2 promoter, an IL-12 promoter, a p40 promoter, and a Bc1-xL promoter.

The compositions and nucleic acid compositions can be administered to subjects or and/delivered into cells by art-known methods or as described herein. Genetic modification of a cell (e.g., a T cell) can be accomplished by transducing a substantially homogeneous cell composition with a recombinant DNA construct. In certain embodiments, a retroviral vector (either a gamma-retroviral vector or a lentiviral vector) is employed for the introduction of the DNA construct into the cell. Non-viral vectors may be used as well.

Possible methods of transduction also include direct co-culture of the cells with producer cells, e.g., by the method of Bregni, et al. (1992) Blood 80:1418-1422, or culturing with viral supernatant alone or concentrated vector stocks with or without appropriate growth factors and polycations, e.g., by the method of Xu, et al. (1994) Exp. Hemat. 22:223-230; and Hughes, et al. (1992) J. Clin. Invest. 89:1817.

Other transducing viral vectors can be used to modify a cell. In certain embodiments, the chosen vector exhibits high efficiency of infection and stable integration and expression (see, e.g., Cayouette et al., Human Gene Therapy 8:423-430, 1997; Kido et al., Current Eye Research 15:833-844, 1996; Bloomer et al., Journal of Virology 71:6641-6649, 1997; Naldini et al., Science 272:263-267, 1996; and Miyoshi et al., Proc. Natl. Acad. Sci. U.S.A. 94:10319, 1997). Other viral vectors that can be used include, for example, adenoviral, lentiviral, and adena-associated viral vectors, vaccinia virus, a bovine papilloma virus, or a herpes virus, such as Epstein-Barr Virus (also see, for example, the vectors of Miller, Human Gene Therapy 15-14, 1990; Friedman, Science 244:1275-1281, 1989; Eglitis et al., BioTechniques 6:608-614, 1988; Tolstoshev et al., Current Opinion in Biotechnology 1:55-61, 1990; Sharp, The Lancet 337:1277-1278, 1991; Cornetta et al., Nucleic Acid Research and Molecular Biology 36:311-322, 1987; Anderson, Science 226:401-409, 1984; Moen, Blood Cells 17:407-416, 1991; Miller et al., Biotechnology 7:980-990, 1989; LeGal La Salle et al., Science 259:988-990, 1993; and Johnson, Chest 107:77S-83S, 1995). Retroviral vectors are particularly well developed and have been used in clinical settings (Rosenberg et al., N. Engl. J. Med 323:370, 1990; Anderson et al., U.S. Pat. No. 5,399,346).

Non-viral approaches can also be employed for genetic modification of a cell. For example, a nucleic acid molecule can be introduced into a cell by administering the nucleic acid in the presence of lipofection (Feigner et al., Proc. Natl. Acad. Sci. U.S.A. 84:7413, 1987; Ono et al., Neuroscience Letters 17:259, 1990; Brigham et al., Am. J. Med. Sci. 298:278, 1989; Staubinger et al., Methods in Enzymology 101:512, 1983), asialoorosomucoid-polylysine conjugation (Wu et al., Journal of Biological Chemistry 263:14621, 1988; Wu et al., Journal of Biological Chemistry 264:16985, 1989), or by micro-injection under surgical conditions (Wolff et al., Science 247:1465, 1990). Other non-viral means for gene transfer include transfection in vitro using calcium phosphate, DEAE dextran, electroporation, and protoplast fusion. Liposomes can also be potentially beneficial for delivery of DNA into a cell. Transplantation of normal genes into the affected tissues of a subject can also be accomplished by transferring a normal nucleic acid into a cultivatable cell type ex vivo (e.g., an autologous or heterologous primary cell or progeny thereof), after which the cell (or its descendants) are injected into a targeted tissue or are injected systemically.

Polynucleotide therapy methods can be directed from any suitable promoter (e.g., the human cytomegalovirus (CMV), simian virus 40 (SV40), or metallothionein promoters), and regulated by any appropriate mammalian regulatory element or intron (e.g. the elongation factor 1a enhancer/promoter/intron structure). For example, if desired, enhancers known to preferentially direct gene expression in specific cell types can be used to direct the expression of a nucleic acid. The enhancers used can include, without limitation, those that are characterized as tissue- or cell-specific enhancers. Alternatively, if a genomic clone is used as a therapeutic construct, regulation can be mediated by the cognate regulatory sequences or, if desired, by regulatory sequences derived from a heterologous source, including any of the promoters or regulatory elements described above.

The resulting cells can be grown under conditions similar to those for unmodified cells, whereby the modified cells can be expanded and used for a variety of purposes.

### Kits

The presently disclosed subject matter provides kits for inducing and/or enhancing an immune response and/or treating and/or preventing a cancer or a pathogen infection in a subject. In certain embodiments, the kit comprises an effective amount of presently disclosed cells or a pharmaceutical composition comprising thereof. In certain embodiments, the kit comprises a sterile container; such containers can be boxes, ampules, bottles, vials, tubes, bags, pouches, blister-packs, or other suitable container forms known in the art. Such containers can be made of plastic, glass, laminated paper, metal foil, or other materials suitable for holding medicaments. In certain non-limiting embodiments, the kit includes an isolated nucleic acid molecule encoding a presently disclosed HR template.

If desired, the cells and/or nucleic acid molecules are provided together with instructions for administering the cells or nucleic acid molecules to a subject having or at risk of developing a cancer or pathogen or immune disorder. The instructions generally include information about the use of the composition for the treatment and/or prevention of a cancer or a pathogen infection. In certain embodiments, the instructions include at least one of the following: description of the therapeutic agent; dosage schedule and administration for treatment or prevention of a neoplasia, pathogen infection, or immune disorder or symptoms thereof; precautions; warnings; indications; counter-indications; over-dosage information; adverse reactions; animal pharmacology; clinical studies; and/or references. The instructions may be printed directly on the container (when present), or as a label applied to the container, or as a separate sheet, pamphlet, card, or folder supplied in or with the container. The resulting cells can be grown under conditions similar to those for unmodified cells, whereby the modified cells can be expanded and used for a variety of purposes.

It will be readily apparent to those skilled in the art that other suitable modifications and adaptations of the methods described herein may be made using suitable equivalents without departing from the scope of the embodiments disclosed herein. Having now described certain embodiments in detail, the same will be more clearly understood by reference to the following examples, which are included for purposes of illustration only and are not intended to be limiting. The scope of the disclosure is indicated by the appended claims rather than by the foregoing description, and all changes that come within the meaning and range of equivalency of the claims are therefore intended to be embraced herein.

### EXAMPLES

The following are examples of methods and compositions of the invention. It is understood that various other embodiments may be practiced, given the general description provided above.

### Example 1

### Materials and Methods

Patients, specimen collection and response assessment. Patients with metastatic melanoma were selected as they signed and informed consent to collect PBMC and tumour biopsies while receiving therapy with anti-PD-1 therapy alone or in combination with other drugs. Additional biopsies and PBMC samples were longitudinally collected if patient condition allowed. Matched cell lines (n=5), PBMCs (n=11) and TILs (n=7) were used in this study. Clinical response was assessed following RECIST criteria (Wolchok, et al. Clinical Cancer Research 15, 7412-7420 (2009). A summary of the patient characteristics is shown below in Table 2.

TABLE 2
Patient Characteristics

| Patient | | | | Site of | | Sites of | | Best response | Duration of |
|---|---|---|---|---|---|---|---|---|---|
| ID | Gender | Race | Age | origin | Stage | metastases | Treatment | on therapy | response |
| PT1 | Male | Caucasian | 79 | Skin | M1b | Lung | Nivolumab | PR | 30+ months, |
| | | | | (scalp) | | | | | ongoing |
| PT2 | Male | Caucasian | 69 | Unknown | M1a | Lymph | Nivolumab | PR | 23+ months, |
| | | | | primary | | nodes | | | ongoing |
| PT3 | Female | Caucasian | 73 | Skin | M1c | Lymph nodes, | Nivolumab* | PD | 0 months |
| | | | | (ankle) | | adrenal gland, | | | |
| | | | | | | kidney, bone | | | |
| PT4 | Female | Asian | 83 | Mucosal | M1c | Lung, | Pembrolizumab | PD | 3 months |
| | | | | (nose) | | liver | | | |
| PT5 | Male | Caucasian | 63 | Skin | M1c | Subcutaneous | Pembrolizumab | SD | 17 months, |
| | | | | (torso) | | tissue, | | | followed by |
| | | | | | | adrenal | | | progression |

PBMC purification, TILs expansion, and cell line establishment. PBMCs from 5-20 mL blood samples were purified using Ficoll-Hypaque (GE Healthcare) gradient separation utilizing SepMate™-50 tube (Stem Cell Technologies). After purification, PBMCs were cryopreserved in freezing media (FBS (Omega Scientific)+10% DMSO (Sigma-Aldrich)), and stored in liquid nitrogen. Cell lines and TIL cultures were established from core needle biopsies of metastatic lesions. TIL cultures were established using tumour fragments as previously described (Dudley, et al., Journal of immunotherapy 26, 332-342 (2003)) and cryopreserved in either CTL Cryo ABC Freezing Kit (ImmunoSpot) or CryoStor CS10 (StemCell Technologies). TIL cultures were established from baseline and on-therapy biopsies. To establish cell lines the tissue was minced with disposable scalpels to generate a single cell suspension and maintained in the tissue culture plates with DMEM 10% human AB serum (Omega scientific, Inc) and supplemented with antibiotics and L-Glutamine until the cells start growing. We consider a cell line is established when there is no evidence of contaminating residual fibroblasts and at least after 10-15 passages. Cell lines were only established from baseline biopsies.

Cell lines and cell culture. All melanoma cell lines (M202, M495, M486, M488, M489 and M490) were maintained in RPMI supplemented with 10% foetal bovine serum (Omega Scientific, Inc), antibiotics, and L-glutamine and kept at 37° C. in a humidified atmosphere of 5% CO₂. Cell lines were periodically authenticated using short-tandem repeat analysis (GenePrint® 10 System, Promega, analysis was outsourced to Laragen Inc.) and tested for mycoplasma presence (Mycoalert, Mycoplasma detection kit, Lonza). For cytotoxicity studies, all cell lines were lentivirally transduced with a lentiviral vector expressing nuclear red fluorescent protein (nRPF) under the control of the EF1a promoter (Essen Bioscience) following the manufacturer's indications, expanded, and sorted using FACS ARIA or FACS ARIA-H (BD bioscience).

Identification of tumour somatic coding mutations and neoantigen selection. Tumour cell lines derived from patient tumour biopsies and matched PBMC were subjected for whole exome sequencing (WES). In addition, RNA-Sequencing (RNA-Seq) was performed on tumour cell lines. DNA and RNA were isolated from cell lines (1 million cells) and PBMCs (1-3 million cells) using Qiagen DNeasy Blood & Tissue Kit (Qiagen) and the Qiagen RNeasy kit (Qiagen) following manufacturer instructions. Whole-exome and RNA sequencing was performed at the Technology Center for Genomics & Bioinformatics (TCGB) core at UCLA using the Illumina Hiseq3000 platform with read length of 2×150 bp paired end. Libraries for whole exome sequencing were generated using Nimblegen SeqCap EZ Human Exome Library v3.0 (Roche). Poly-A selection was used for RNAseq library construction.

Neoepitope prediction and ranking was performed. Briefly, first, WES sequences were aligned to human hg19 reference genome. Somatic coding mutations identified by both VarScan2 and MuTect were retained as potential neoantigens. Second, RNA-Seq sequences were mapped to human hg19, quantified and normalized with HISAT2 and StringTie. Third, the neoantigen sequences and patient's HLA types identified from patient's PBMC WES were used as input for HLA-peptide binding affinity prediction with netMHCpan. Finally, the HLA-peptide complexes with predicted binding affinities among top 2% ranking with respect to each HLA were selected and only the peptides with confirmed expression by RNA-Seq in those selected complexes were proceeded to protein reagent generation.

Preparation of HLA-peptide complex libraries. HLA-peptide complex libraries were generated as described in PCT/US2019/025415 (which is herein incorporated by reference in its entirety). Briefly, NeoE encoding DNA fragments were cloned into modified pcDNA3.1 vector encoding for a soluble HLA allele in 96 well format. DNA clones were verified by SANGER sequencing and plasmids were complexed with ExpiFectamine™ (Thermo Fisher) and transfected into HEK293F cells using manufacturer recommendation. Secreted Neo-HLA complexes were biotinylated enzymatically with BirA ligase (Brand) and purified from clarified cell culture media by IMAC (Brand). Purified protein libraries were buffer exchanged by desalting chromatography (brand) and protein concentration was measured by Absorbance at 280 nm.

Patient neoepitope-specific T-cell identification. Neoepitope-specific T-cell identification was performed as described in PCT/US2020/17887 (which is herein incorporated by reference in its entirety). Briefly, each neoepitope-HLA element was DNA barcoded and multimerized by two different fluorescent streptavidins (phycoerythrin-PE- or allophycocyanin-APC-). The DNA barcode followed the structure: Universal primer 1-NNNNN-Barcode-NNNNNN-Universal primer 2. The barcoded and fluorescently multimerized neoepitope-HLA elements were pooled together to stain PBMC and TILs. Incubation of the barcoded and multimerized neoepitope-HLA library with patient PBMCs or TILs was followed by fluorescent-activated cell sorting (FACS). Dual fluorescently-labelled (PE+APC) multimer-bound CD8 T cells were sorted as single cells into individual wells of plates. These wells were then subjected to Reverse transcription and PCR to amplify the T-cell receptor (TCR) and barcode DNA sequences. The sequences were then subject to next-generation sequencing by Illumina mini-seq machines to decode the TCR and barcode information.

Due to non-specific binding events, not all dual fluorescent positive cells are neoepitope-specific. By analyzing DNA barcodes, neoepitope-specific T cells would predominantly have the same barcode, while non-specific T cells would have different barcodes. TCR sequences from neoepitopes-specific T cells were selected and used to genetically modify healthy donor T cells as described below.

Homology Directed Repair (HDR) template generation. Generation of HDR templates was performed as described in PCT/US2018/058230 (which is herein incorporated by reference in its entirety). The TCR paired variable regions from neoepitope-specific T cells isolated in the previous step were amplified by PCR and purified. The purified PCR products were assembled with constant regions and homology arms to generate the patient-specific HDR templates. The patient-specific HDR templates were verified through Sanger sequencing and agarose gel electrophoresis.

Cell Editing and neoepitope-specificity validation. Non-viral T-cell gene editing was performed as described in PCT/US2018/058230 (which is herein incorporated by reference in its entirety). Primary human T cells were isolated from healthy donor's enriched leukapheresis using magnetic affinity selection. The isolated T cells were activated with anti-CD3/CD28 particles and cultured for 48-72 hours. After activation, the T cells were centrifuged and resuspended in P3 buffer (Lonza). Ribonucleoproteins (RNPs) were formulated by complexing guide RNAs targeting TRAC and TRBC to Cas9 protein. The patient-specific HDR template and RNPs were mixed with the cell suspension and electroporated, and subsequently transferred into complete T cell medium. To confirm the neoepitope-specificity of the cloned TCRs, gene edited T cells were stained with neoepitope-HLA dextramers (30 nM, manufactured in-house as described above).

T-cell functional studies: T-cell activation, cytokine release, proliferation, degranulation, and cytotoxicity. T-cell activation, cytokine release, and proliferation were measured upon co-culture with autologous (M485, M486, M488, M489 and M490) or mismatched (M202) cell lines. Briefly, melanoma cells (25×10³ cells/well for T-cell proliferation and cytokine release or 30×10³ for T-cell activation) were seeded in 96-well plates, and incubated overnight either with media alone or, when indicated, media supplemented with IFNγ (2000 IU/mL, millipore). Then, melanoma cells were washed and neoTCR gene edited T cells were added with a product:target (P:T) of 5:1.

To measure cytokine release, co-culture supernatants were collected at 24 h and stored at −80° C. When ready for analysis, the supernatants were thawed at room temperature and the concentration of IFNγ, TNFα, IL2 and IL6 was measured using a cytokine bead array (Human Th1/Th2 Cytokine kit, BD bioscience). Data is shown normalized by edited cells.

To measure T-cell activation, after 20-24 h co-culture T cells were collected, wash with PBS, and stained with Zombie Violet (1:100, Biolegend) live/dead stain in PBS. After incubation, cells were stained with neoepitope-HLA dextramers-PE (30 nM, manufactured as indicated above) and then with CD45-FITC clone HI30 (BD Bioscience), OX40-PECy7 clone Ber-ACT35, 4-1BB-APC clone 4B4-1, CD4-BV501 clone OKT4, and CD8-BV605 clone RPA-T8 (all from Biolegend) antibodies at the concentrations recommended by the manufacturers.

To measure T-cell proliferation, after 24, 48, and 72 h co-culture T cells were collected, wash, and stained with Live/Dead NIR fluorescent reactive (1:250 Invitrogen). After incubation, cells were washed, stained with neoepitope-HLA dextramers-PE (30 nM, manufactured as indicated above), and then with CD4-AlexaFluor700 clone SK3 (1:400, BD Biosciences), and CD8-BV605 clone RPA-T8 (1:50, Biolegend). After incubation, cells were then washed, permeabilized with fixation/permeabilization working solution (Foxp3/Transcription Factor Staining Buffer Set, ThermoFisher), washed with permeabilization buffer (Foxp3/Transcription Factor Staining Buffer Set, ThermoFisher) and stained with Ki67-BV421 clone B56 (1:100, BD Biosciences) antibody.

T-cell degranulation was measured upon 12-16 h co-culture with autologous or mismatched cell lines. 50×10³ melanoma cells were seeded in 48-well plates and incubated 8 h with either media alone or media supplemented with IFNg (2000 IU/mL, Millipore). After incubation, melanoma cells were washed and 250×10³ neoTCR gene edited T cells were added (P:T=5:1) together with CD107a-APC-H7 clone H4A3 (BD) at the recommended concentration. After 1 h incubation, brefeldin A and monesin (BD Golgi Plug and BD Golgi stop, BD Biosciences) were added at half the recommended concentration to inhibit the protein transport. Cells were incubated for 11-15 h more and then surface expression of CD107a was measured by flow cytometry. Briefly, T cells were collected and stained with Zombie Violet Live/Dead, neoepitope-HLA dextramer-APC, CD45-FITC clone HI30, CD107a-APC-H7 clone H4A3 BD (both from BD Bioscience), CD4-BV501 clone OKT4, and CD8-BV605 clone RPA-T8 (both from Biolegend).

In all flow cytometry stainings, after the last incubation with antibodies, cells are then washed, fixed and stored at 4° C. until flow cytometry acquisition. All stains and washes were performed in stain buffer (BD bioscience) unless otherwise indicated. Flow cytometry acquisition was performed on an Attune NxT flow cytometer (Invitrogen).

To measure cytotoxicity, 25×10³ cells/well of the nRFP melanoma cell lines were seeded in 96-well plates, and incubated overnight either with media alone or, when indicated, media supplemented with IFNγ (2000 IU/mL, Millipore). Then, melanoma cells were washed and neoTCR gene edited T cells were added with P:T rations ranging from 10:1 to 1:1. After adding the T cells, melanoma cells were imaged using a real-time live cell imaging system (Incucyte, Essen Biosciences) and followed for at least 150 h.

NeoTCR gene edited T cells targeting an irrelevant mutation in these cell lines (Neo12) were used as a control in all experiments. All functional studies were done in melanoma cell media, not supplemented with cytokines. T-cell activation, degranulation, proliferation and cytokine release were done in biological triplicates. Cytotoxicity experiments were done in biological quadruplicates unless otherwise stated.

Software. FlowJo software was used to analyse flow cytometry data, Graphpad Prism used for data representation and statistical analysis, RAWgraph was used for circle packing visualizations.

Statistics. In T-cell functionality studies, unpaired t test with Holm-Sidak adjustment for multiple comparisons was used to compare between neo-epitope specific TCR and the neo12 TCR control or between autologous and mismatched control cell lines.

### Example 2

### NeoTCR Products are Effective for Treating Non-Responder Patients

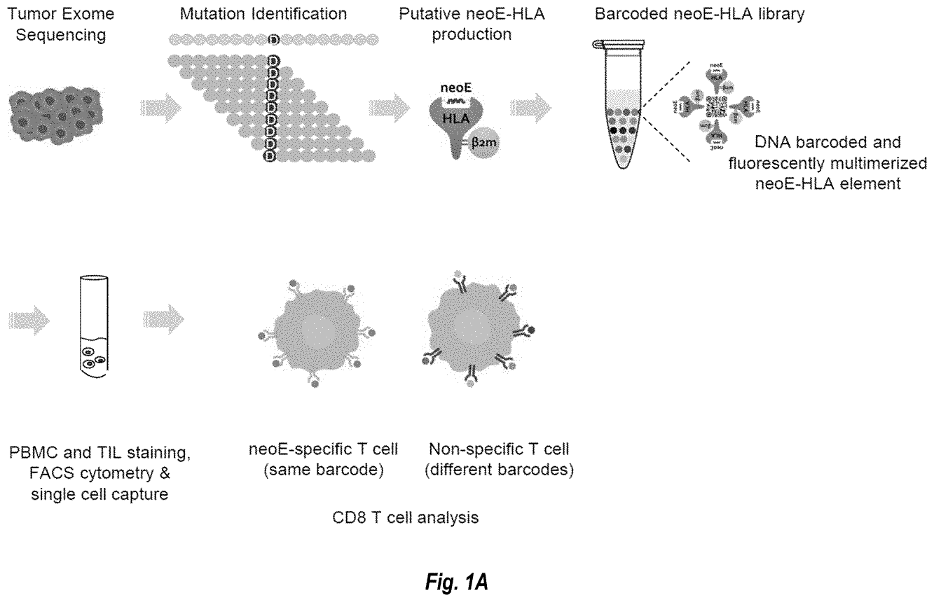

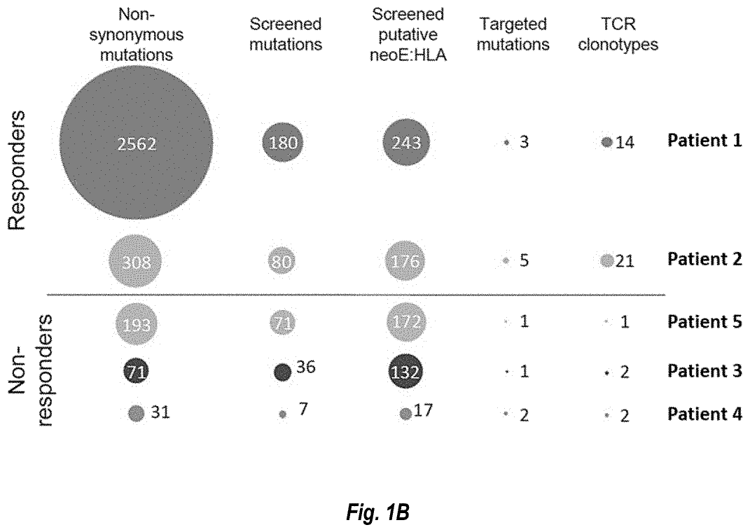

To characterize the neoepitope-specific T cell responses induced after checkpoint inhibitor therapy, peripheral blood mononuclear cells (PBMCs) were collected over time (longitudinally) and established expanded tumor infiltrating lymphocyte cultures (TILs) and autologous tumor cell lines from the patient's tumor biopsies. Whole exome and RNA sequencing was performed on the tumor and normal tissue controls for the computational prediction and ranking of patient-specific neoepitopes. A library of capture reagents consisting of the patient HLA class I molecules loaded with predicted neoepitopes was generated (see Peng et al. AACR 2019) and isolated neoepitope-specific T cells from the patients' PBMC or TIL samples. Once isolated, the paired neoepitope-specific TCR alpha and beta chains (NeoTCRs) from isolated T cells were obtained by single cell sequencing. This data was used to define each patient's six HLA class I alleles, detect patient-specific melanoma non-synonymous mutations and to perform computational predictions of the resulting putative neoepitopes. The putative neoepitopes were prioritized based on their level of expression by the tumour and the predicted binding affinity to the patient's own HLA class I alleles. After selection, tens to hundreds of soluble putative neoepitope-HLA class I proteins were produced by single chain trimer technology. These personalized protein libraries were then DNA-barcoded and fluorescently multimerized for use as capture reagents. Once assembled, the libraries were incubated with patients' PBMC or lymphocytes expanded from tumour biopsies, and the neoepitope-specific T cells were captured by single cell sorting. The barcodes unique to each putative neoepitope-HLA multimers were then used to decipher the antigen specificity of the captured T cells (FIG. 1A).

For functional characterization of the NeoTCRs, healthy donor primary human T cells were modified to express the neoTCR using CRISPR-based, non-viral precision genome engineering by replacing the endogenous TCR with the respective NeoTCR (Jacoby et al., AACR 2019, Sennino et al., AACR 2019). These gene-edited T cells were then used in co-culture experiments with the patient autologous cell lines.

T cell responses were analyzed in five patients (PT1, PT2, PT3, PT4, and PT5) with metastatic melanoma receiving checkpoint inhibitor therapy. Two patients (PT1 and PT2) had longstanding partial responses to nivolumab infusions that are ongoing at over two years from the start of therapy, with only residual lesions that did not uptake ¹⁸fluorodeoxyglucose by positron emitting tomography (PET) imaging, suggesting that they had no active melanoma. PT3 progressed rapidly prior to the planned first infusion of nivolumab. PT4 progressed after receiving pembrolizumab for four months. PT5 received a combination of pembrolizumab and intratumoural injection of the toll like receptor agonist 9 (TLR9) SD101, resulting in regression of the subcutaneous site that received SD101 injections and stable disease of adrenal metastases that lasted for 17 months, followed by widespread progression of bone and peritoneal metastases. See Table 2.

Whole exome sequencing of the patient-derived melanoma cell lines, identified a wide range of mutations, ranging from 2562 in patient PT1 to 31 in PT4 (median 193, see Table 3, below). The two patients with response to anti-PD-1 therapy had a higher mutational tumor load than the three patients without a response to therapy. Despite the wide range of expressed mutations, the number of mutations recognized as neoepitopes by the T cells was a lot less variable, ranging from 1 to 5 (FIG. 1B; Table 3). Therefore, there was evidence of immunodominance, i.e. the T cell responses to mutational neoantigens were not linearly correlated with the number of mutations but seem to be focused on a limited set of them that were recognized as neoepitopes, both in patients with and without a clinical response to anti-PD-1 therapy. However, single cell TCR sequencing of neoepitope-specific T cells isolated from blood and tumor from patients PT1 and PT2 (who had a clinical response to therapy), revealed that they had multiple individual TCR alpha and beta chain pairs (TCR clonotypes), 14 and 21 clonotypes, respectively. This is opposed to the neoepitope-specific T cells isolated from the three patients without a response to therapy, which had two, two, and one TCR clonotypes, respectively (FIG. 1B and Table 3).

Subsequently, longitudinal analysis of the neoepitope recognition for each patient was performed in all the available sequential blood and tumor samples and the functionality of the isolated TCRs was tested against the autologous melanoma cell lines.

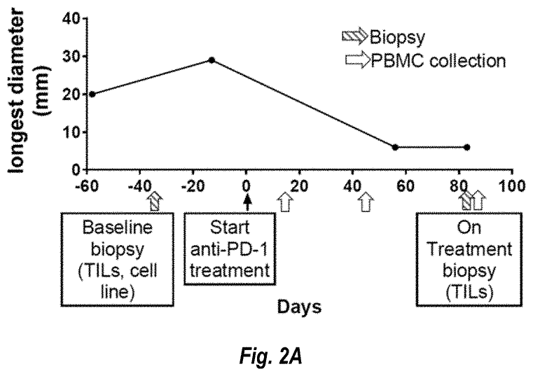

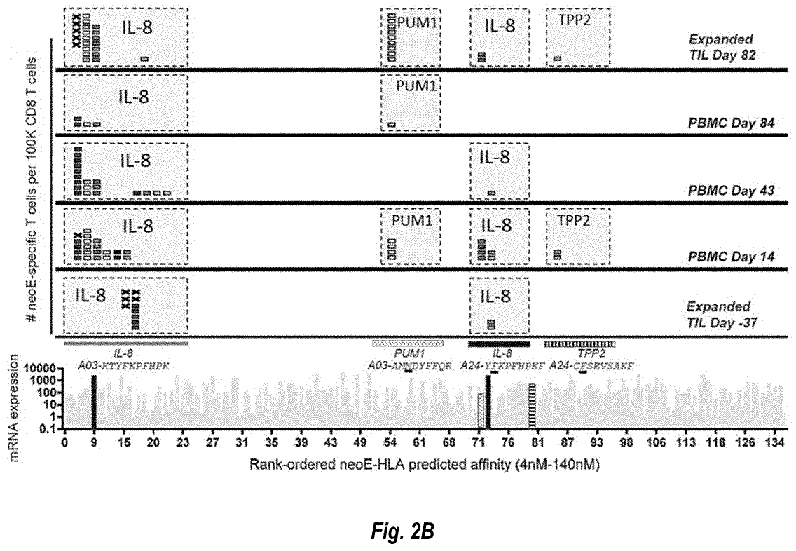

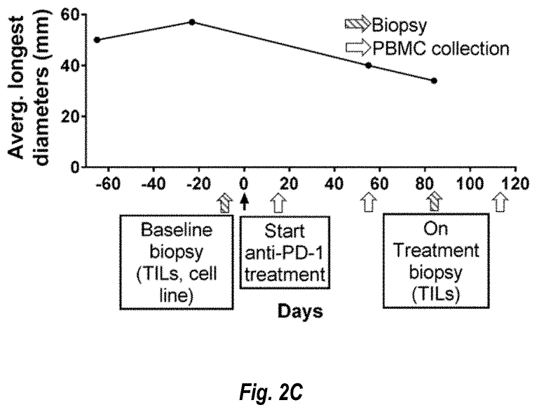

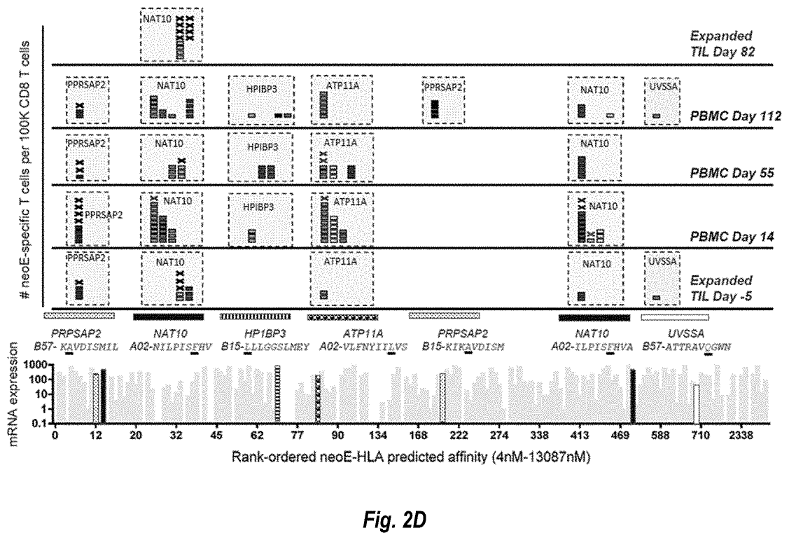

PT1 had a fast and durable anti-tumor response to anti-PD-1 therapy in lung metastases (FIG. 2A). Sequencing identified 2562 somatic coding mutations, of which 1099 were predicted to be expressed by RNA sequencing. A library of 243 neoepitope-specific pMHC capture reagents covering 183 mutations across 3 HLA types, HLA-A03:01, A24:01 and C12:03 was generated and used for screening CD8 T cells from PBMCs or TILs derived from multiple longitudinal time points. 186 neoepitope-specific T cells were isolated that comprised 14 different TCR clonotypes targeting only three mutations. Notably, the same mutations were recognized as neoepitopes at most of the time points analyzed, supporting the immunodominance hypothesis. See Tables 2 and 3. The 14 neoepitope-specific TCRs (neoTCRs) were detected in the sequential blood and tumor samples with different quantities, some expanding or contracting at different timepoints. It should be noted that the peripheral blood draw was between 5 and 20 milliliters and the tumor biopsies were core needle biopsies, which only sampled a small minority of T cells in each compartment at any time point.

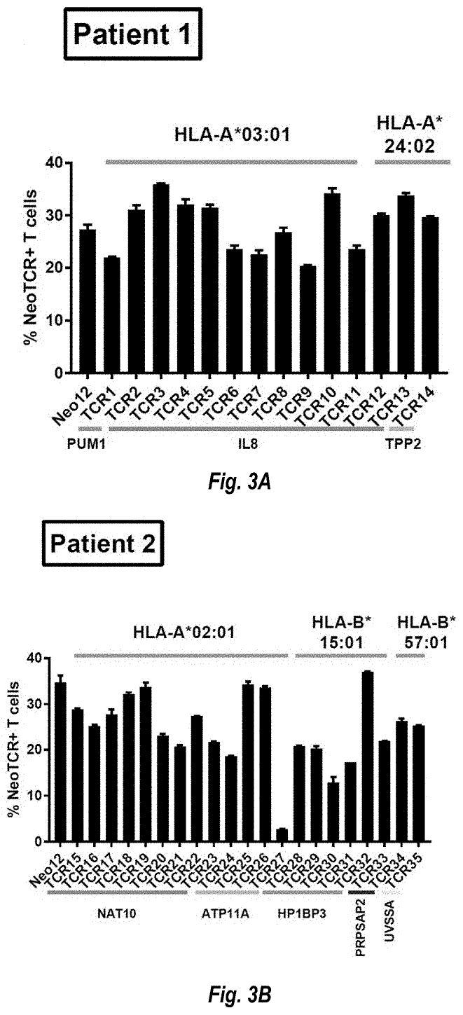

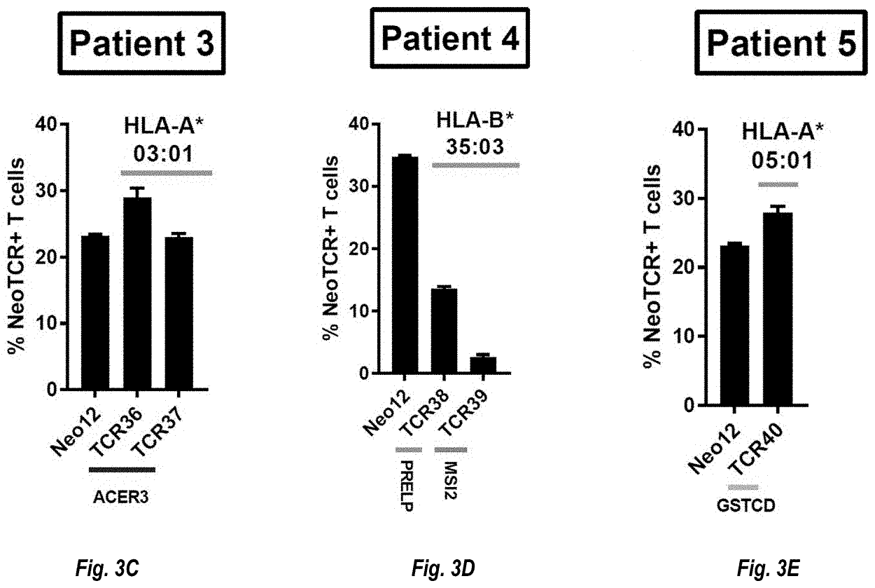

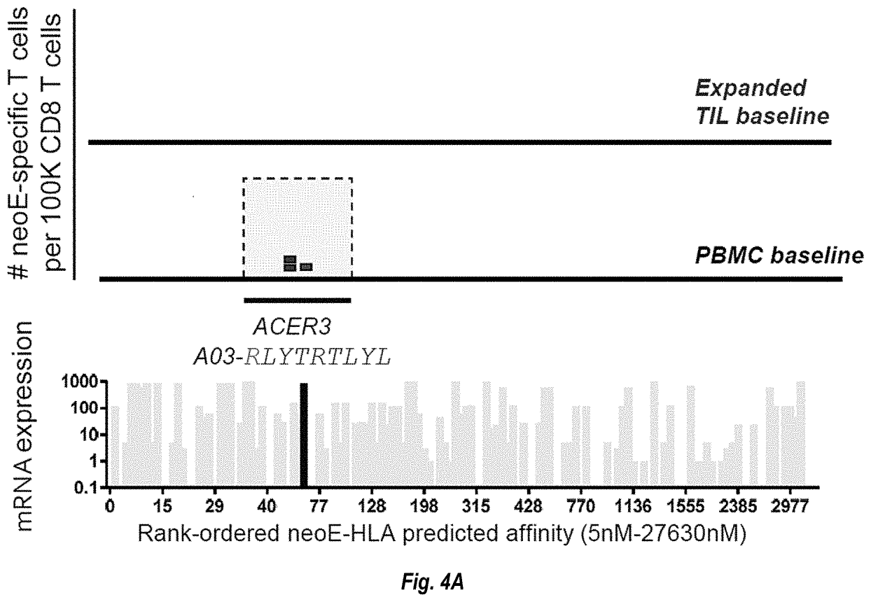

Similar findings were observed in samples from PT2, who had a long-lasting response to anti-PD-1 therapy in lymph node metastasis from melanoma (FIG. 2C). In this case, out of the 308 somatic coding mutations, 126 were expressed (Table 3). The patient-specific library contained 176 capture reagents covering 80 mutations presented by the six HLAs class I, and 296 neoepitope-specific T cells were isolated targeting five mutations; 21 different neoTCR clonotypes targeting these mutations were validated (FIG. 2D, Tables 3 and 4, and FIGS. 3A-3E).

On the other hand, PT4, PT3, and PT5 showed marginal responses to checkpoint inhibitors and were classified as Non-Responder Patients.

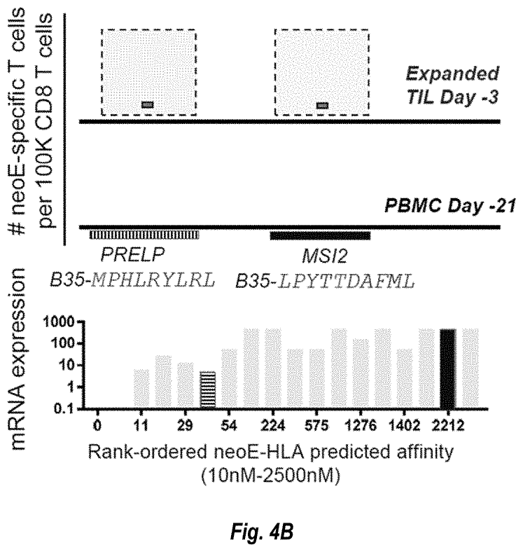

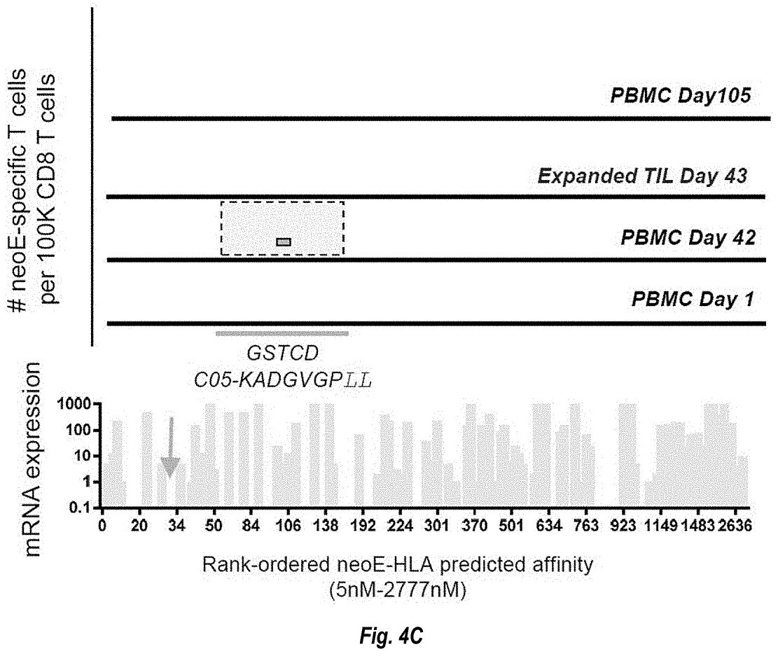

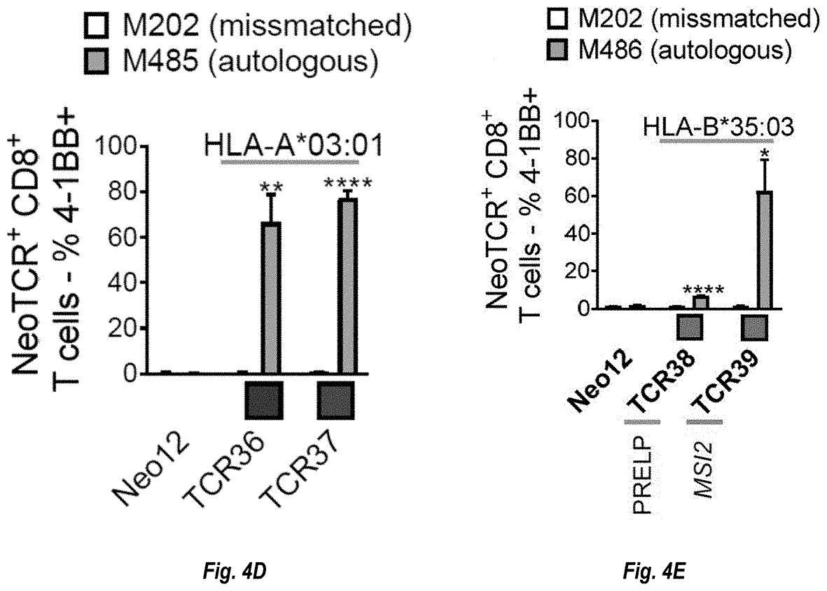

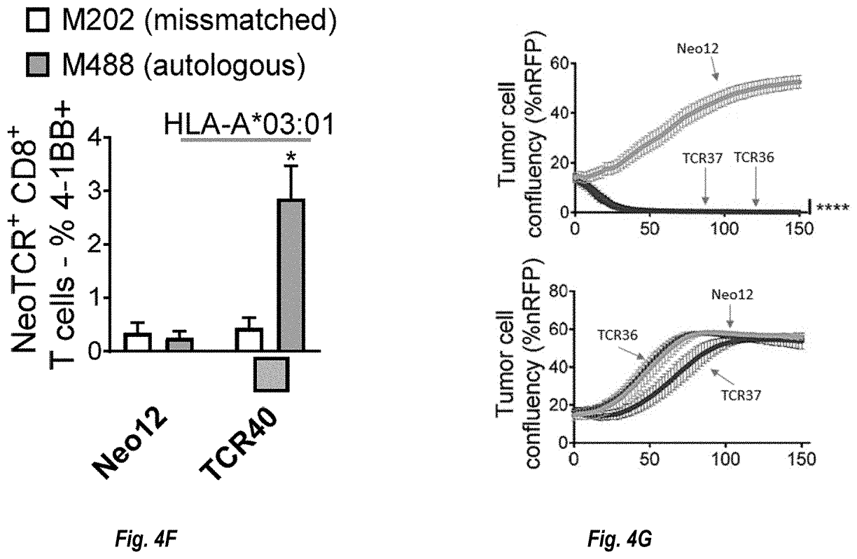

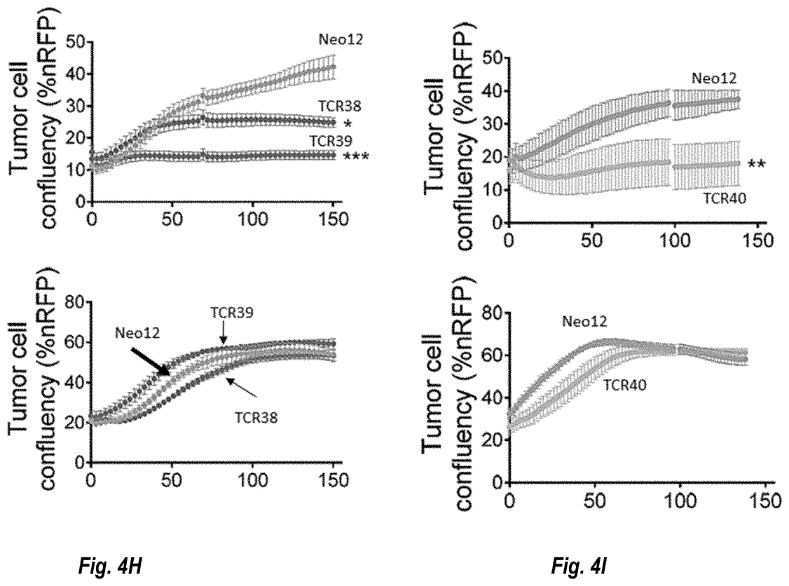

The melanoma cell line from PT3 had 71 non-synonymous mutations, 33 of which were expressed. A library of 132 capture reagents covering 36 mutations presented by five HLAs was prepared and used for capture neoepitope-specific T cells from baseline blood and tumor (Table 3). Of note, several of the mutations did not have mRNA expression detected by sequencing. Two different clonotypes of T cells were identified in the blood targeting the same mutation in ACER3 presented by HLA-A*03:01 (FIG. 4A and Tables 3 and 4). The melanoma cell line from PT4 had 31 total non-synonymous mutations and 20 of them were expressed. The library contained 17 capture reagents covering seven mutations presented by three HLA class I (Table 3). Two neoepitope-specific T cells in the tumour that targeted mutations in the PRELP and MSI2 (FIG. 4B and Tables 3 and 4) were identified. The melanoma cell line from PT5 had 193 non-synonymous mutations of which 61 were expressed. A library of 172 capture reagents was assembled covering 71 mutations presented by the six HLAs class I. One neoTCR clonotype of neoepitope-specific T cells in one out of four blood or tumour samples was identified, which targeted a mutation in GSTCD presented by HLA-C*05:01 (FIG. 4C and Tables 3 and 4). Therefore, mutational neoepitope-specific T-cell responses from patients who did not respond to anti-PD-1 therapy were oligoclonal and not recurrent in blood and tumor samples over time.

Table 3 shows a summary of each patient's mutations and targeted neoepitopes.

TABLE 3
Patient mutations and targeted neoepitopes

| | | Non- | | | | | | | |
|---|---|---|---|---|---|---|---|---|---|
| Patient | Cell | synonymous | Expressed | # capture | # mutations | | | HLA-Neoepitope | # TCR |
| ID | line | mutations | mutations | reagents | screened | HLAs covered | Gene | sequence | clonotype |
| PT1 | M489 | 2562 | 1099 | 243 | 180 | HLA-A*03:01 | IL8 | A03-KTYFKPFHPK | 10 |
| | | | | | | HLA-A*24:02 | | A24-YFKPFHPKF | 2 |
| | | | | | | HLA-C*12:03 | PUM1 | A03-AMMDYFFQR | 1 |
| | | | | | | | TPP2 | A24-CFSEVSAKF | 1 |
| PT2 | M490 | 308 | 126 | 176 | 80 | HLA-A*01:01 | NAT10 | A02-NILPISFHV | 5 |
| | | | | | | HLA-A*02:01 | | A02-ILPISFHVA | 4 |
| | | | | | | HLA-B*15:01 | ATP11A | A02-VLFNYIILVS | 4 |
| | | | | | | HLA-B*57:01 | HP1BP3 | B15-LLLGGSLMEY | 5 |
| | | | | | | HLA-C*03:04 | PRPSAP2 | B57-KAVDISMIL | 1 |
| | | | | | | HLA-C*06:02 | | B15-KIKAVDISM | 1 |
| | | | | | | | UVSSA | B57-ATTRAVQGWN | 1 |
| PT3 | M485 | 71 | 33 | 132 | 36 | HLA-A*02:01 | ACER3 | A03-RLYTRTLYL | 2 |
| | | | | | | HLA-A*03:01 | | | |
| | | | | | | HLA-B*07:02 | | | |
| | | | | | | HLA-C*05:01 | | | |
| | | | | | | HLA-C*07:02 | | | |
| PT4 | M486 | 31 | 20 | 17 | 7 | HLA-B*35:03 | PRELP | B35-MPHLRYLRL | 1 |
| | | | | | | HLA-C*08:01 | MSI2 | B35-LPYTTDAFML | |
| | | | | | | HLA-C*12:03 | | | 1 |
| PT5 | M488 | 193 | 61 | 172 | 71 | HLA-A*01:01 | GSTCD | C05-KADGVGPLL | 1 |
| | | | | | | HLA-A*02:01 | | | |
| | | | | | | HLA-B*27:05 | | | |
| | | | | | | HLA-B*44:02 | | | |
| | | | | | | HLA-C*04:01 | | | |
| | | | | | | HLA-C*05:01 | | | |

To functionally test the neoepitope specificity of the isolated TCRs, the paired neoTCR alpha and beta chains of captured individual T cells were sequenced and used to genetically modify healthy donor T cells using non-viral CRISPR/Cas9 gene editing methods to replace the endogenous TCR with the neoepitope-specific TCRs. For this process, guide RNAs targeting the first exon of the TCR-alpha constant region (TRAC) locus, DNA homology directed repair templates encoding for the full neoTCR beta chain and the variable neoTCR alpha chain, and Cas9 protein were electroporated into previously activated T cells. After homology-directed repair, a fully functional neoTCR was integrated in the genome under the control of endogenous TCR alpha regulation. As a validation step, the antigen specificity of the resulting neoTCR transgenic T cells was confirmed by dextramer staining (FIGS. 3A-3E).

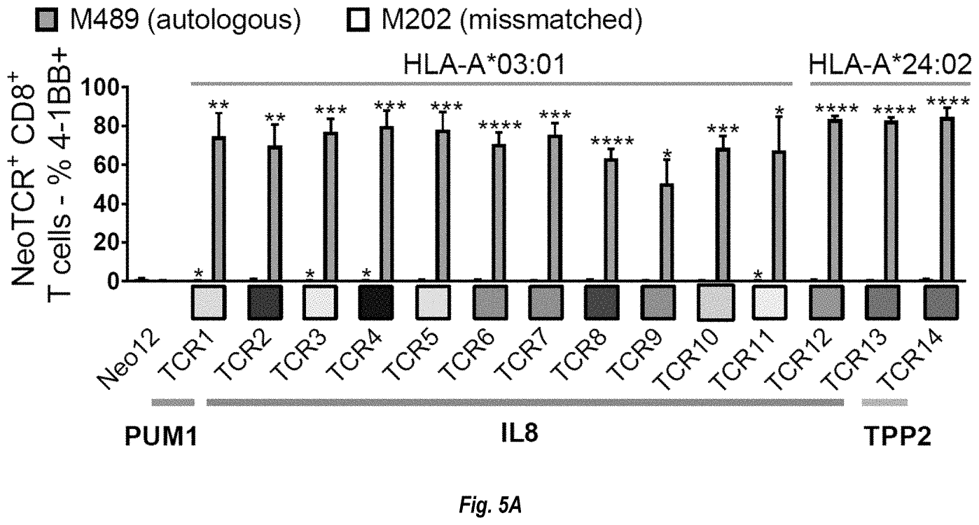

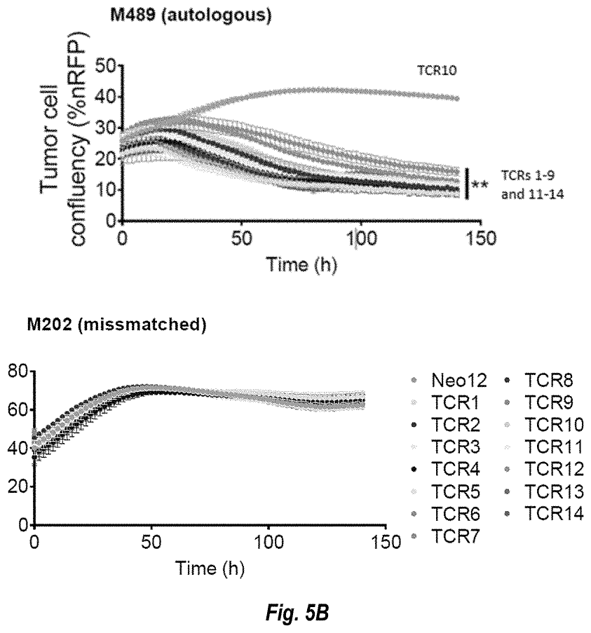

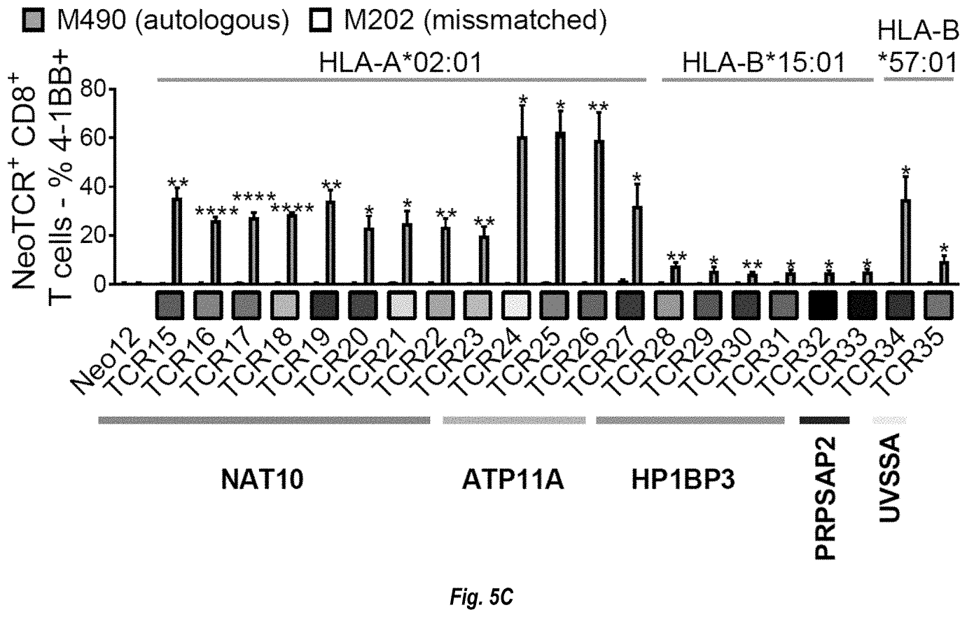

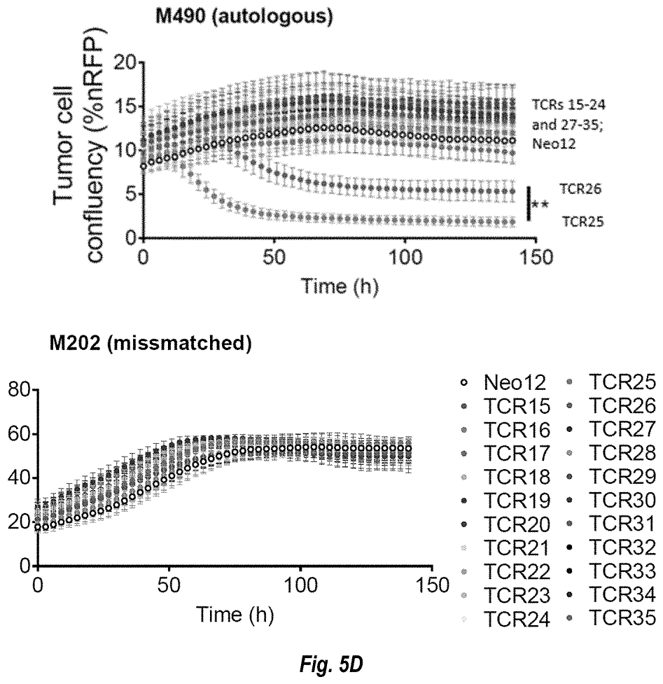

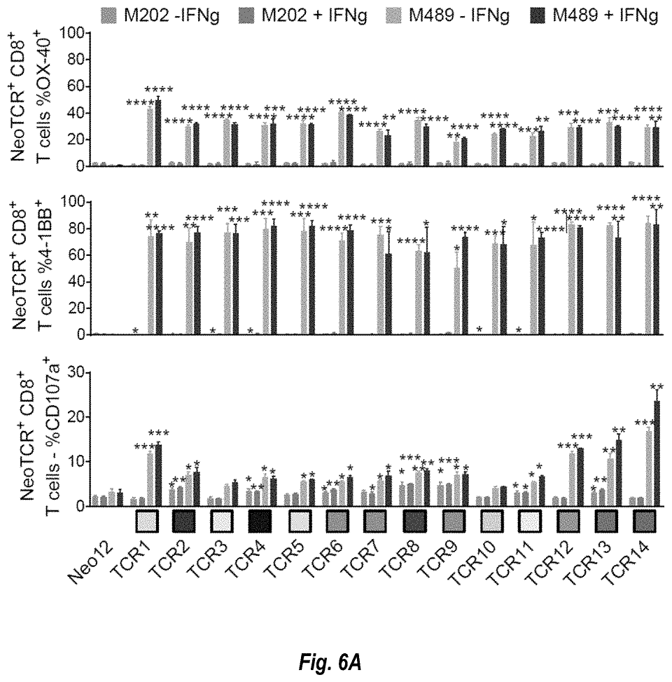

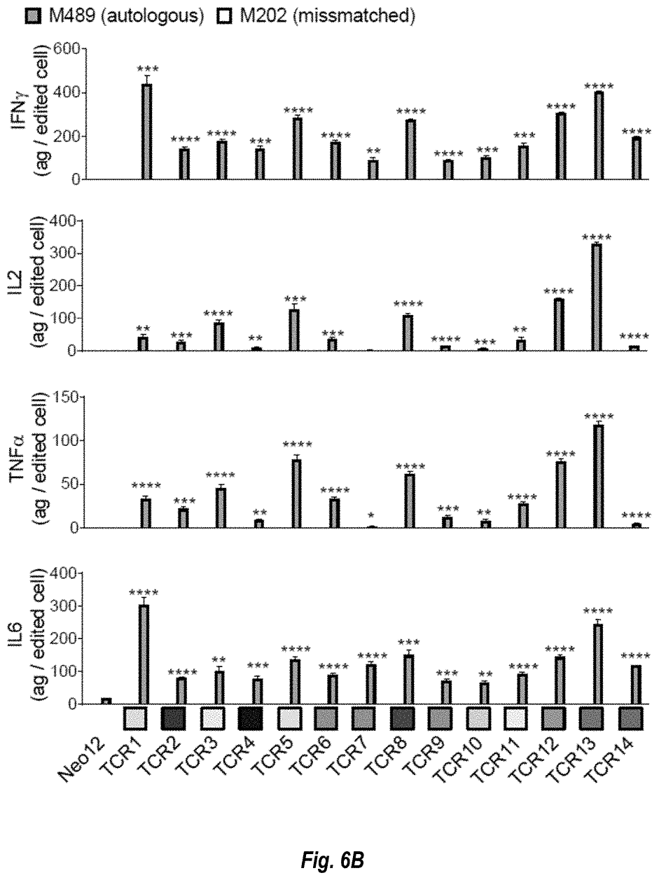

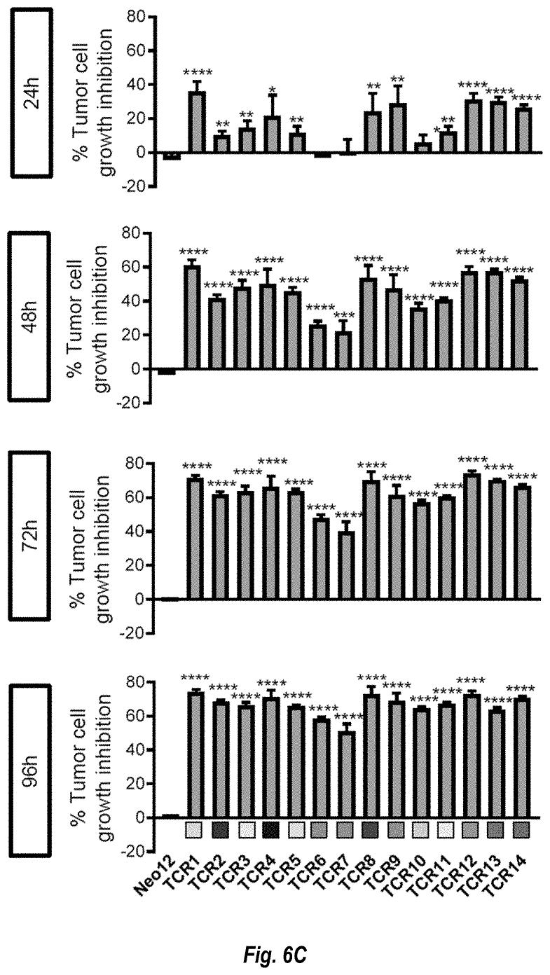

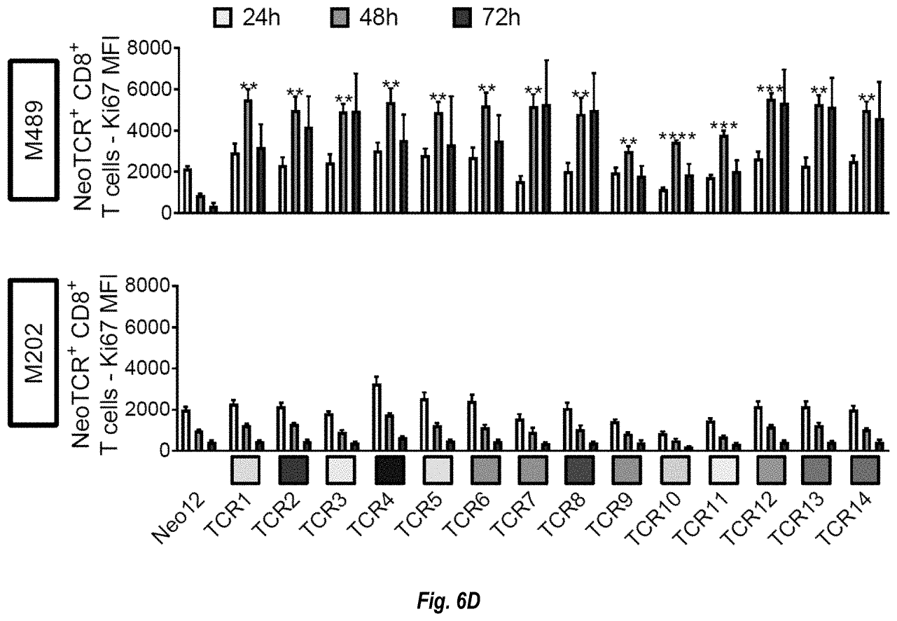

For PT1, fourteen (14) different neoTCRs specific for neoepitopes in the mutated IL8, PUM1 and TPP2 genes were characterized. Co-culture experiments with the neoTCR gene edited T cells and a cell line established from the patient's baseline biopsy (M489), or an unmatched cell line (M202) were performed. A neoTCR isolated from another patient, targeting a mutation irrelevant in these cell lines, Neo12, was used as a negative control. Each of these fourteen (14) NeoTCR Products displayed specific cytotoxicity against the matched autologous melanoma cell line established from a biopsy of PT1 (50-75% tumor growth inhibition compared to melanoma cell line growth in co-culture with a mismatched control TCR, 96 hour assay using a product to target ratio (P:T) of 1:1, p<0.000001 for each comparison). No cytotoxic effect against an unmatched human melanoma cell line was observed (FIG. 5B and FIG. 6C). Furthermore, NeoTCR Products upregulated 4-1BB and OX-40, which are surface markers on T cells reflective of recent TCR engagement. The 14 neoTCR transgenic T cells released interferon gamma (IFNγ), interleukin-2 (IL-2), tumor necrosis factor alpha (TNFα), and IL6, and displayed T-cell degranulation and proliferation upon co-culture with the patient-matched melanoma cell line. Again, no unspecific T cell activation was observed when NeoTCR Products were co-cultured with unmatched targets.

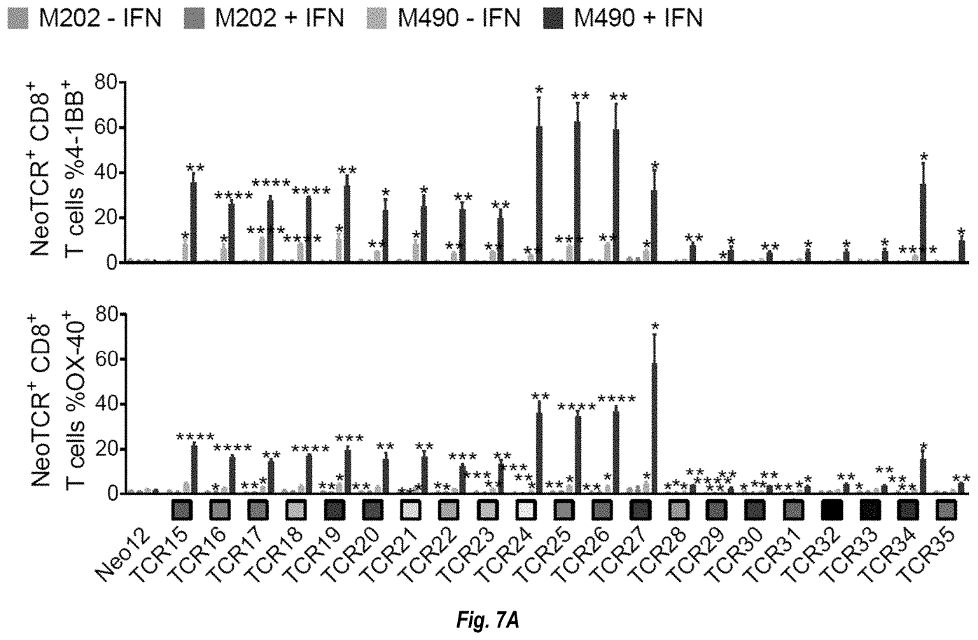

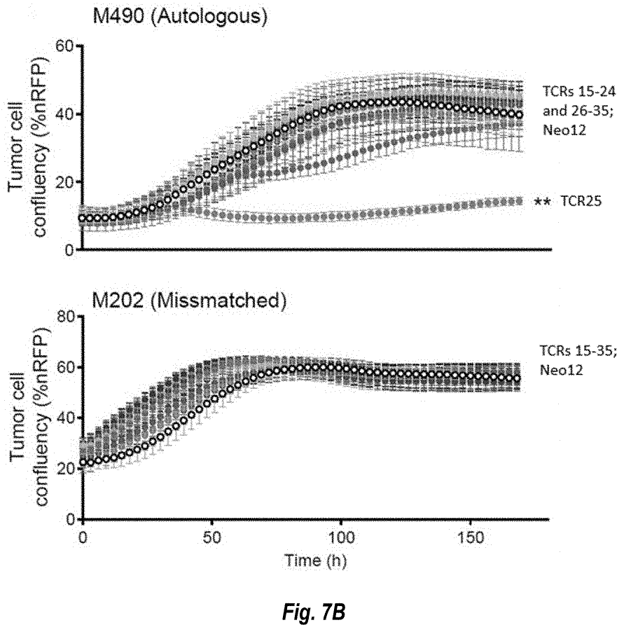

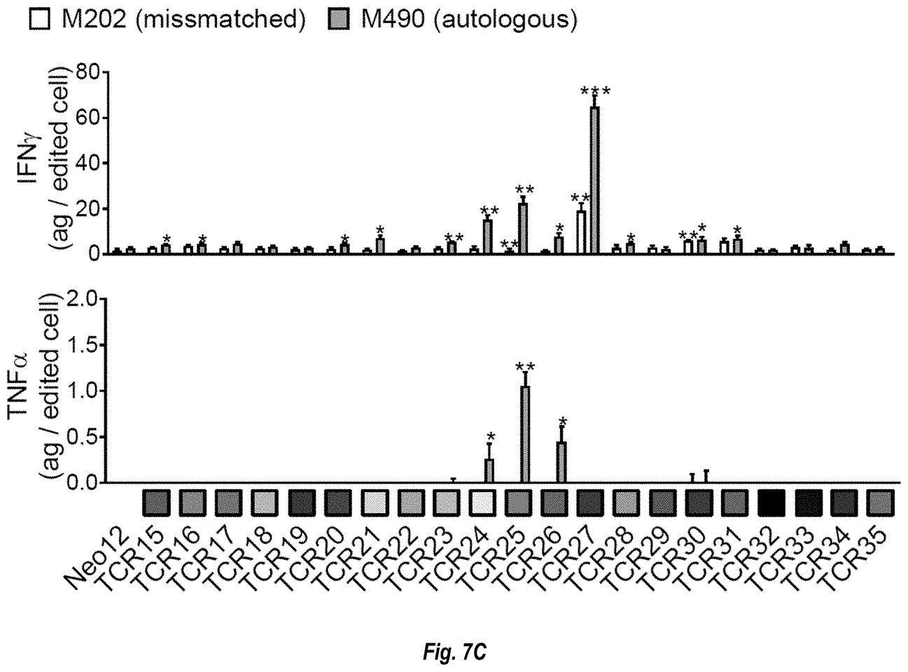

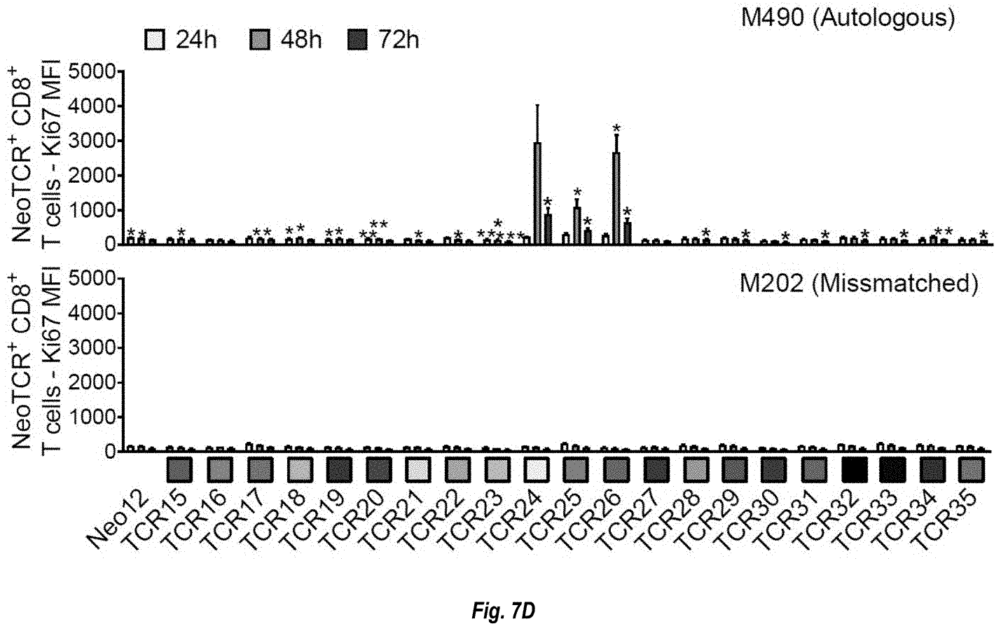

Similarly, the 21 neoTCR clonotypes isolated from PT2 were selected for generation of the corresponding neoTCR T-cell products that targeted point mutations in NAT10, ATP11A, HP1BP3, PRPSAP2, and UVSSA presented by the six HLA class I of this patient. Initial attempts at testing these neoTCRs for T-cell activation, cytokine responses, and cytotoxicity upon co-culture with the patient-specific melanoma cell line (M490) showed very little activity, which was surprising given the good clinical response to PD-1 blockade therapy in this patient. It was hypothesized that the presentation of the neoepitopes may be low in the M490 cell line and may need to be upregulated by pre-exposure to interferon-gamma (IFNγ). When this step was added, CD8⁺ T cells expressing the 21 neoTCRs isolated from this patient resulted in upregulation of the surface expression of 4-1BB (FIG. 5C). TCRs 24, 25 and 26 targeting the mutation in ATP11A secreted IFNγ and TNFα, and induced T-cell proliferation (FIGS. 7C-7D). Additionally, TCR25 induced specific cytotoxicity to the matched melanoma cell line even without IFNγ pre-exposure, while TCR25 and TCR26 induced cytotoxicity with IFNγ pre-exposure (FIG. 5D and FIG. 7B).

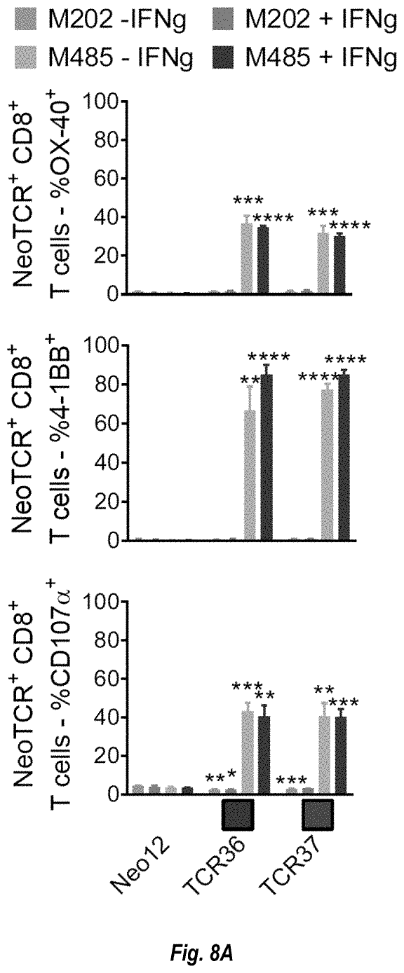

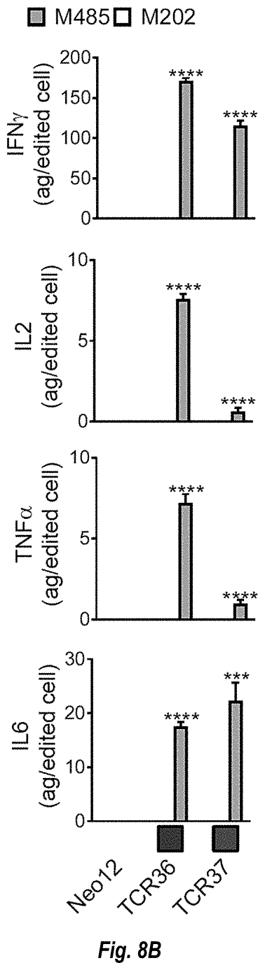

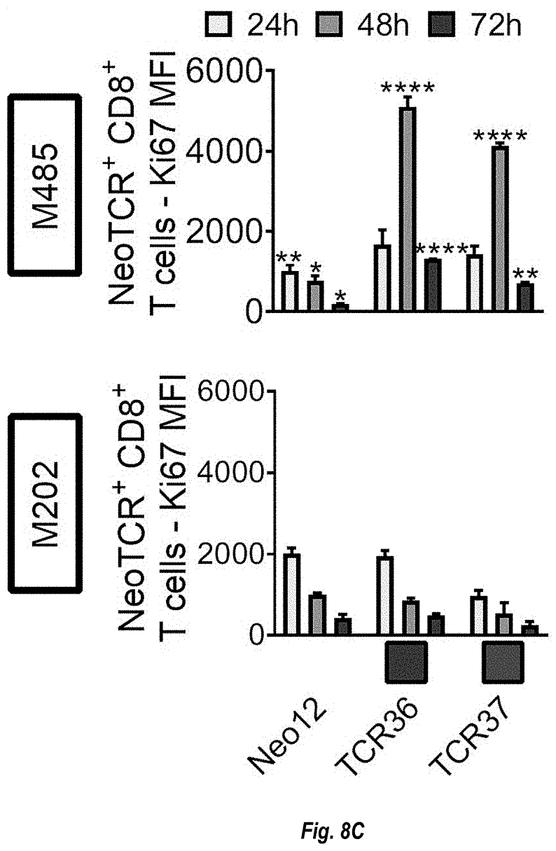

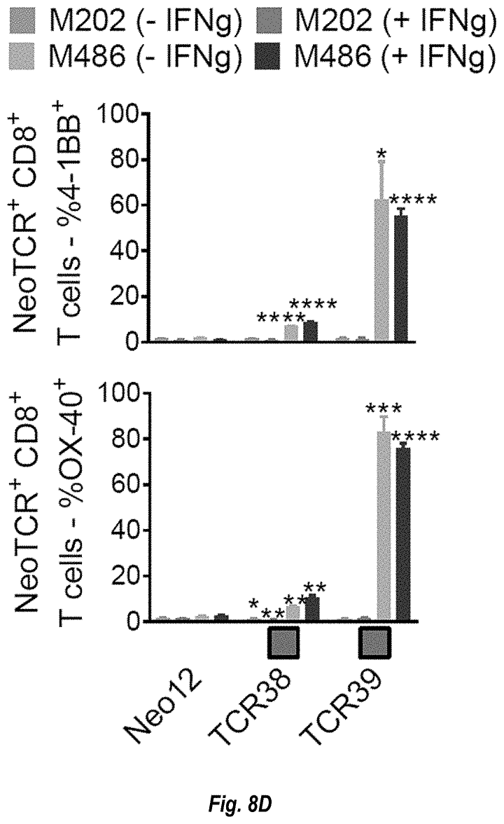

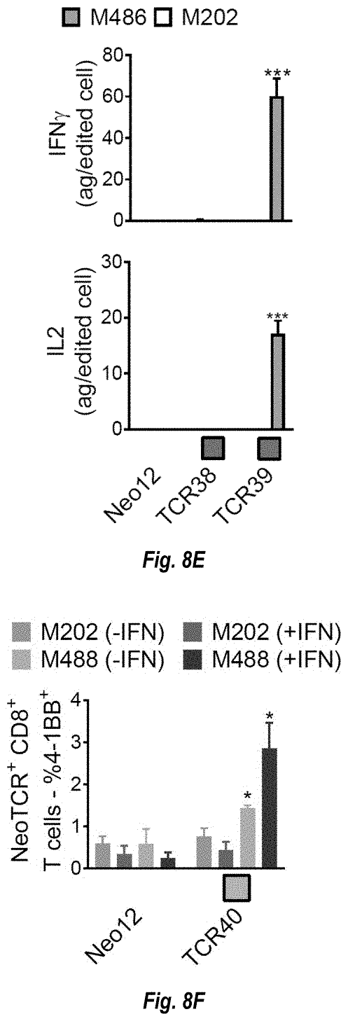

NeoTCR gene edited T-cell products from the three patients without clinical response to anti-PD-1 were analyzed for functional specificity against their corresponding autologous melanoma cells lines established from biopsies from these patients (FIGS. 4D-4I and FIG. 8). T cells expressing neoTCRs isolated from patients PT4, PT3, and PT5 recognized the matched melanoma cell line and upregulated the 4-1BB and OX40 activation markers upon co-culture (FIGS. 4D and 4F and FIGS. 8A, 8D and 8F). TCR36 and TCR37 from PT3, and TCR39 from PT4, also induced secretion of IFNγ, TNFα, IL2, and IL6 and proliferation (FIGS. 8B and 8E). T cells expressing all neoTCRs displayed specific cytotoxicity against the corresponding autologous melanoma cell lines (35-100% tumor growth inhibition compared to melanoma cell line growth in co-culture with a mismatched control TCR, 96 hour assay using P:T 5:1, p<0.05 for each comparison), without having an effect on the mismatched control cell lines (FIGS. 4G-4I).

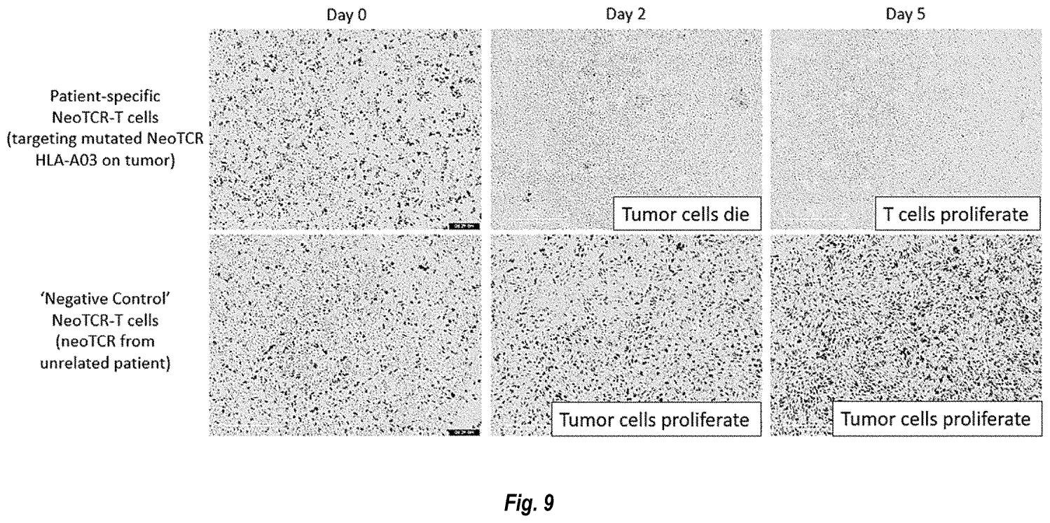

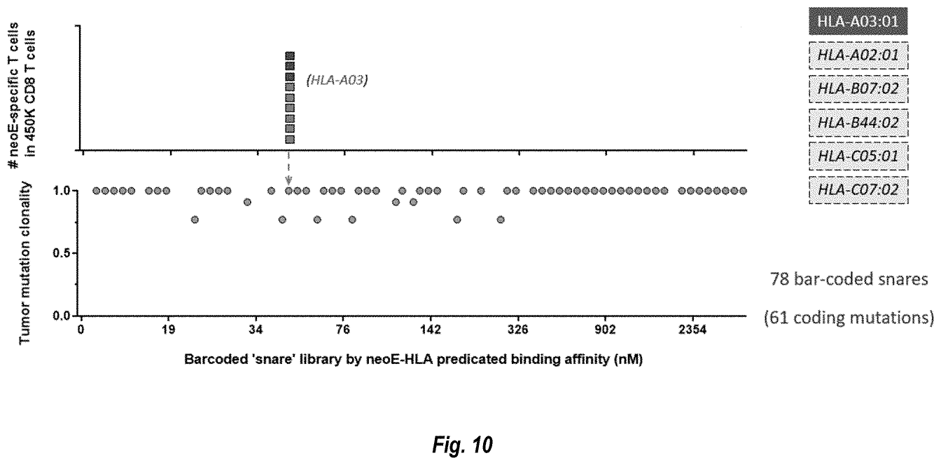

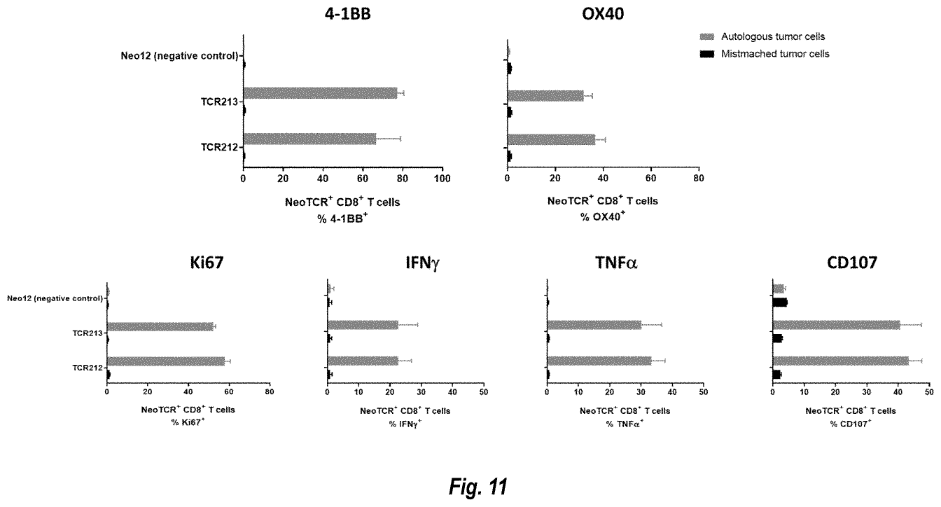

Additional support for the data presented in FIGS. 4A-4I is shown in FIG. 9. FIG. 9 shows a cancer cell death from a Non-Responder Patient in response to patient-specific NeoTCR Products. The top row of images shows that a NeoTCR Product designed for a Non-Responder Patient kills cancer cells and also that the neoTCR T cells proliferated. In order to show that the cell death was due to the NeoTCR Product, a negative control was performed and shown in the bottom row of images. In the bottom row of images, the Neo12 NeoTCR Product that does not correlate to a neoepitope on the Non-Responder Patient's cancer cells does not kill the Non-Responder Patient's cancer cells. More so, the longer the Non-Responder Patient's cancer cells were kept in culture with the Neo12 NeoTCR Product negative control, the more the cancer cells proliferated.

Table 4 summarizes the neoepitope-specific T-cell isolation.

TABLE 4
Neoepitope-specific T-cell isolation

| | | | | | | | | Expanded |
|---|---|---|---|---|---|---|---|---|
| | | | | | | | PBMCs | TILs |
| | | | | Expanded TILs | PBMCs Day 14 | PBMCs Day 43 | Day 84 | Day 82 |
| | | | | Day −37 (90K | (50K cells | (160K cells | (100K cells | (310K cells |
| PT1 | Gene | Peptide | HLA | cells analyzed) | analyzed) | analyzed) | analyzed) | analyzed) |
| TCR1 | PUM1 | AMMDYFFQR | A*03:01 | 0 | 2 | 0 | 1 | 9 |
| TCR2 | IL8 | KTYFKPFHPK | A*03:01 | 0 | 7 | 13 | 5 | 0 |
| TCR3 | IL8 | KTYFKPFHPK | A*03:01 | 0 | 1 | 0 | 0 | 0 |
| TCR4 | IL8 | KTYFKPFHPK | A*03:01 | 0 | 1 | 0 | 0 | 0 |
| TCR5 | IL8 | KTYFKPFHPK | A*03:01 | 0 | 3 | 4 | 2 | 59 |
| TCR6 | IL8 | KTYFKPFHPK | A*03:01 | 0 | 1 | 0 | 0 | 0 |
| TCR7 | IL8 | KTYFKPFHPK | A*03:01 | 0 | 2 | 4 | 2 | 7 |
| TCR8 | IL8 | KTYFKPFHPK | A*03:01 | 49 | 0 | 1 | 0 | 0 |
| TCR9 | IL8 | KTYFKPFHPK | A*03:01 | 0 | 0 | 1 | 0 | 1 |
| TCR10 | IL8 | KTYFKPFHPK | A*03:01 | 0 | 0 | 1 | 0 | 0 |
| TCR11 | IL8 | KTYFKPFHPK | A*03:01 | 0 | 0 | 1 | 0 | 0 |
| TCR12 | IL8 | YFKPFHPKF | A*24:02 | 1 | 1 | 1 | 0 | 0 |
| TCR13 | IL8 | YFKPFHPKF | A*24:02 | 0 | 2 | 0 | 0 | 2 |
| TCR14 | TPP2 | CFSEVSAKF | A*24:02 | 0 | 1 | 0 | 0 | 1 |
| | | | | | | | | Expanded |
| | | | | | | | PBMCs | TILs |
| | | | | Expanded TILs | PBMCs Day 14 | PBMCs Day 55 | Day 112 | Day 82 |
| | | | | Day −5 (181K | (37K cells | (48K cells | (115K cells | (92K cells |
| PT2 | Gene | Peptide | HLA | cells analyzed) | analyzed) | analyzed) | analyzed) | analyzed) |
| TCR15 | NAT10 | NILPISFHV | A*02:01 | 0 | 2 | 0 | 2 | 0 |
| TCR16 | NAT10 | NILPISFHV | A*02:01 | 0 | 7 | 0 | 5 | 0 |
| TCR17 | NAT10 | NILPISFHV | A*02:01 | 0 | 1 | 1 | 1 | 0 |
| TCR18 | NAT10 | NILPISFHV | A*02:01 | 58 | 0 | 6 | 0 | 77 |
| TCR19 | NAT10 | NILPISFHV | A*02:01 | 4 | 0 | 0 | 4 | 0 |
| TCR20 | NATI0 | ILPISFHVA | A*02:01 | 2 | 10 | 2 | 3 | 0 |
| TCR21 | NAT10 | ILPISFHVA | A*02:01 | 0 | 1 | 0 | 0 | 0 |
| TCR22 | NATI0 | ILPISFHVA | A*02:01 | 0 | 4 | 0 | 0 | 0 |
| TCR23 | NATI0 | ILPISFHVAT | A*02:01 | 0 | 0 | 0 | 1 | 0 |
| TCR24 | ATP11A | VLFNYIILVS | A*02:01 | 0 | 2 | 1 | 0 | 0 |
| TCR25 | ATP11A | VLFNYIILVS | A*02:01 | 2 | 7 | 11 | 6 | 0 |
| | | | | | | | | Expanded |
| | | | | | | | PBMCs | TILs |
| | | | | Expanded TILs | PBMCs Day 14 | PBMCs Day 55 | Day 112 | Day 82 |
| | | | | Day −5 (181K | (37K cells | (48K cells | (115K cells | (92K cells |
| PT2 | Gene | Peptide | HLA | cells analyzed) | analyzed) | analyzed) | analyzed) | analyzed) |
| TCR26 | ATP11A | VLFNYIILVS | A*02:01 | 0 | 1 | 0 | 0 | 0 |
| TCR27 | ATP11A | VLFNYIILVS | A*02:01 | 0 | 0 | 1 | 0 | 0 |
| TCR28 | HP1BP3 | LLLGGSLMEY | B*15:01 | 0 | 1 | 0 | 1 | 0 |
| TCR29 | HP1BP3 | LLLGGSLMEY | B*15:01 | 0 | 0 | 1 | 0 | 0 |
| TCR30 | HP1BP3 | LLLGGSLMEY | B*15:01 | 0 | 0 | 1 | 0 | 0 |
| TCR31 | HP1BP3 | LLLGGSLMEY | B*15:01 | 0 | 0 | 0 | 1 | 0 |
| TCR32 | HP1BP3 | LLLGGSLMEY | B*15:01 | 0 | 0 | 0 | 1 | 0 |
| TCR33 | PRPSAP2 | KIKAVDISM | B*15:01 | 0 | 0 | 0 | 4 | 0 |
| TCR34 | PRPSAP2 | KAVDISMIL | C*03:04 | 23 | 16 | 10 | 13 | 0 |
| TCR35 | UVSSA | ATTRAVQGWN | B*57:01 | 1 | 0 | 0 | 1 | 0 |
| | | | | Expanded TILs | PBMCs baseline | | | |
| | | | | baseline (200K | (400K cells | | | |
| PT3 | Gene | Peptide | HLA | cells analyzed) | analyzed) | | | |
| TCR36 | ACER3 | RLYTRTLYL | A*03:01 | 0 | 6 | — | — | — |
| TCR37 | ACER3 | RLYTRTLYL | A*03:01 | 0 | 3 | — | — | — |
| | | | | Expanded TILs | PBMCs Day −21 | | | |
| | | | | Day −3 (400K | (400K cells | | | |
| PT4 | Gene | Peptide | HLA | cells analyzed) | analyzed) | | | |
| TCR38 | PRELP | MPHLRYLRL | B*35:03 | 1 | 0 | — | — | — |
| TCR39 | MSI2 | LPYTTDAFML | B*35:03 | 1 | 0 | — | — | — |
| | | | | | | | PBMCs | |
| | | | | PBMCs Day 1 | PBMCs Day 42 | Expanded TILs | Day 105 | |
| | | | | (200K cells | (250K cells | Day 43 (100K | (100K cells | |
| PT5 | Gene | Peptide | HLA | analyzed) | analyzed) | cells analyzed) | analyzed) | |
| TCR40 | GSTCD | KADGVGPLL | C*05:01 | 0 | 1 | 0 | 0 | — |

### Example 3

### Non-Responder Patients and Immunodominance

Using newly developed techniques to isolate and capture neoE-specific single T cells, as well as non-viral gene editing, neoepitope-specific T cells that can recognize the cancer cells and induce an anti-tumor response were isolated and characterized. The neoepitope immunodominance and TCR clonality over time of the natural T cell repertoire was also studied. It was shown that the neoepitope immunodominance and TCR clonality over time induced anti-tumor responses to checkpoint inhibitor therapy. The results show that in a patient with a good response to anti-PD-1, there is a polyclonal response that targets a limited number of neoepitope-HLA complexes (2% of the neoE tested in the case of patient one) highlighting the immunodominance of these epitopes.

Interestingly, different T cell clonotypes targeting the same mutations evolve over time, suggesting functional differences amongst the TCRs.

It was unexpected to find that Non-Responder Patients harbor neoE-specific T cells. Furthermore, it was discovered that it was possible to isolate neoepitope-specific T cells and make a NeoTCR Product that was able to recognize and kill patient-derived cancer cells. This shows that neoepitope-specific TCRs can be isolated from Non-Responder Patients and can be used for personalized adoptive T cell therapies such as NeoTCR Products.

### Example 4

### NeoTCR Products Kill Patient Specific Cancer Cells

Two patient specific NeoTCR Products (TCR36 (also referred to as TCR212) and TCR37 (also referred to as TCR213)) were generated for a Non-Responder Patient. An off-target NeoTCR Product (Neo12) was used as a negative control. Neo12 is a NeoTCR Product that is specific to a different patient that does not have the same neoepitope signatures as the Non-Responder Patient of this example. The TCR36 and TCR37 NeoTCR Products were both effective at 1) killing the Non-Responder Patient tumor cells, 2) preventing growth and expansion of the Non-Responder Patient tumor cells, and 3) decreasing tumor cell confluency of the Non-Responder Patient tumor cells. As a negative control, the Neo12 NeoTCR Product which does not correlate to neoepitope on the Non-Responder Patient's tumor cells, did not kill the Non-Responder Patient's tumor cells and did not prevent cancer cell proliferation in the Non-Responder Patient's tumor cell samples.

An additional control was performed. Specifically, the Neo12, TCR36, and the TCR37 NeoTCR Products were cocultured with a tumor cell line derived from a patient other than the Non-Responder Patient for whom the TCR36 and TCR37 NeoTCR Products were designed and generated. As shown, the TCR36 and TCR37 NeoTCR Products are patient specific and do not kill mismatched tumor cells.

Taken together, it was shown that patient specific NeoTCR Products can be made for Non-Responder Patients and such NeoTCR Products are effective at killing Non-Responder Patients' cancer cells.

Accordingly, NeoTCR Products can be designed, made, and used to treat Non-Responder Patients by specifically killing the Non-Responder Patients' cancer cells.

### Example 5

### Neoepitopes can be Detected in Non-Responder Patients

Non-Responder Patients often have low tumor mutational burdens. Based on this known fact, it was expected that neoepitopes would be difficult, if not impossible, to detect in Non-Responder Patient tumor biopsies. Surprisingly, the methods used here were able to detect neo-TCRs from Non-Responder Patient tumor biopsies and NeoTCR Products that were able to kill the patient cancer cells were able to be made therefrom.

### Example 6

### Generation of NeoTCR Products

Neoepitope-specific TCRs identified by the imPACT Isolation Technology described in PCT/US2020/17887 (which is herein incorporated by reference in its entirety) were used to generate homologous recombination (HR) DNA templates. These HR templates were transfected into primary human T cells in tandem with site-specific nucleases (see FIGS. 12A-12C). The single-step non-viral precision genome engineering resulted in the seamless replacement of the endogenous TCR with the patient's neoepitope-specific TCR, expressed by the endogenous promoter. The TCR expressed on the surface is entirely native in sequence.

The precision of neoTCR-T cell genome engineering was evaluated by Targeted Locus Amplification (TLA) for off-target integration hot spots or translocations, and by next generation sequencing based off-target cleavage assays and found to lack evidence of unintended outcomes.

As shown in FIGS. 12A-12C, constructs containing genes of interest were inserted into endogenous loci. This was accomplished with the use of homologous repair templates containing the coding sequence of the gene of interest flanked by left and right HR arms. In addition to the HR arms, the gene of interest was sandwiched between 2A peptides, a protease cleavage site that is upstream of the 2A peptide to remove the 2A peptide from the upstream translated gene of interest, and signal sequences (FIG. 1B). Once integrated into the genome, the gene of interested expression gene cassette was transcribed as single messenger RNA. During the translation of this gene of interest in messenger RNA, the flanking regions were unlinked from the gene of interest by the self-cleaving 2A peptide and the protease cleavage site was cleaved for the removal of the 2A peptide upstream from the translated gene of interest (FIG. 1C). In addition to the 2A peptide and protease cleavage site, a gly-ser-gly (GSG) linker was inserted before each 2A peptide to further enhance the separation of the gene of interest from the other elements in the expression cassette.

It was determined that P2A peptides were superior to other 2A peptides for Cell Products because of its efficient cleavage. Accordingly, two (2) P2A peptides and codon divergence were used to express the gene of interest without introducing any exogenous epitopes from remaining amino acids on either end of the gene of interest from the P2A peptide. The benefit of the gene edited cell having no exogenous epitopes (i.e., no flanking P2A peptide amino acids on either side of the gene of interest) is that immunogenicity is drastically decreased and there is less likelihood of a patient infused with a Cell Product containing the gene edited cell to have an immune reaction against the gene edited cell.

As described in PCT/US/2018/058230, NeoTCRs were integrated into the TCRα locus of T cells. Specifically, a homologous repair template containing a NeoTCR coding sequence flanked by left and right HR Arms was used. In addition, the endogenous TCRβ locus was disrupted leading to the expression of only TCR sequences encoded by the NeoTCR construct. The general strategy was applied using circular HR templates as well as with linear templates.

The target TCRα locus (Cα) is shown along with the plasmid HR template, and the resulting edited sequence and downstream mRNA/protein products in FIGS. 12B and 12C. The target TCRα locus (endogenous TRAC) and its CRISPR Cas9 target site (horizontal stripe, cleavage site designated by arrow) are shown (FIGS. 12A-12C). The circular plasmid HR template with the polynucleotide encoding the NeoTCR is located between left and right homology arms (“LHA” and “RHA” respectively). The region of the TRAC introduced by the HR template that was codon optimized is shown (vertical stripe). The TCRβ constant domain was derived from TRBC2, which is indicated as being functionally equivalent to TRBC1. Other elements in the NeoTCR cassette include: 2A=2A ribosome skipping element (by way of non-limiting example, the 2A peptides used in the cassette are both P2A sequences that are used in combination with codon divergence to eliminate any otherwise occurring non-endogenous epitopes in the translated product); P=protease cleavage site upstream of 2A that removes the 2A tag from the upstream TCRβ protein (by way of non-limiting example the protease cleavage site can be a furin protease cleavage site); SS=signal sequences (by way of non-limited example the protease cleavage site can be a human growth hormone signal sequence). The HR template of the NeoTCR expression gene cassette includes two flanking homology arms to direct insertion into the TCRα genomic locus targeted by the CRISPR Cas9 nuclease RNP with the TCRα guide RNA. These homology arms (LHA and RHA) flank the neoE-specific TCR sequences of the NeoTCR expression gene cassette. While the protease cleavage site used in this example was a furin protease cleavage site, any appropriate protease cleavage site known to one of skill in the art could be used. Similarly, while HGH was the signal sequence chosen for this example, any signal sequence known to one of skill in the art could be selected based on the desired trafficking and used.

Once integrated into the genome (FIG. 12C), the NeoTCR expression gene cassette is transcribed as a single messenger RNA from the endogenous TCRα promoter, which still includes a portion of the endogenous TCRα polypeptide from that individual T cell (FIG. 12C). During ribosomal polypeptide translation of this single NeoTCR messenger RNA, the NeoTCR sequences are unlinked from the endogenous, CRISPR-disrupted TCRα polypeptide by self-cleavage at a P2A peptide (FIG. 12C). The encoded NeoTCRα and NeoTCRβ polypeptides are also unlinked from each other through cleavage by the endogenous cellular human furin protease and a second self-cleaving P2A sequence motifs included in the NeoTCR expression gene cassette (FIG. 12C). The NeoTCRα and NeoTCRβ polypeptides are separately targeted by signal leader sequences (derived from the human growth hormone, HGH) to the endoplasmic reticulum for multimer assembly and trafficking of the NeoTCR protein complexes to the T cell surface. The inclusion of the furin protease cleavage site facilitates the removal of the 2A sequence from the upstream TCRβ chain to reduce potential interference with TCRβ function. Inclusion of a gly-ser-gly linker before each 2A (not shown) further enhances the separation of the three polypeptides.

Additionally, three repeated protein sequences are codon diverged within the HR template to promote genomic stability. The two P2A are codon diverged relative to each other, as well as the two HGH signal sequences relative to each other, within the TCR gene cassette to promote stability of the introduced NeoTCR cassette sequences within the genome of the ex vivo engineered T cells. Similarly, the re-introduced 5′ end of TRAC exon 1 (vertical stripe) reduces the likelihood of the entire cassette being lost over time through the removal of intervening sequence of two direct repeats.

In-Out PCR was used to confirm the precise target integration of the NeoE TCR cassette. Agarose gels show the results of a PCR using primers specific to the integration cassette and site generate products of the expected size only for cells treated with both nuclease and DNA template (KOKI and KOKIKO), demonstrating site-specific and precise integration.

Furthermore, Targeted Locus Amplification (TLA) was used to confirm the specificity of targeted integration. Crosslinking, ligation, and use of primers specific to the NeoTCR insert were used to obtain sequences around the site(s) of integration. The reads mapped to the genome are binned in 10 kb intervals. Significant read depths were obtained only around the intended site the integration site on chromosome 14, showing no evidence of common off-target insertion sites.

Antibody staining for endogenous TCR and peptide-HLA staining for neoTCR revealed that the engineering results in high frequency knock-in of the NeoTCR, with some TCR-cells and few WT T cells remaining. Knock-in is evidenced by neoTCR expression in the absence of an exogenous promoter. Engineering was carried out multiple times using the same neoTCR with similar results. Therefore, efficient and consistent expression of the NeoTCR and knockout of the endogenous TCR in engineered T cells was achieved.

1. A method of treating cancer in a Non-Responder Patient with a NeoTCR Product, wherein the Non-Responder Patient does not respond to a checkpoint inhibitor therapy or an immunotherapy.

2. A method of killing cancer cells from a Non-Responder Patient with a NeoTCR Product designed and made for such patient, wherein the Non-Responder Patient does not respond to a checkpoint inhibitor therapy or an immunotherapy.

3. (canceled)

4. (canceled)

5. The method of claim 1, wherein the Non-Responder Patient showed no response to a checkpoint inhibitor therapy or an immunotherapy; or initially responded to a checkpoint inhibitor therapy or an immunotherapy, then developed resistance, stopped responding, or showed reduced response.

6. The method of claim 5, wherein the Non-Responder Patient did not respond to a CTLA-4 Binding Agent therapy.

7. (canceled)

8. The method of claim 6, wherein the CTLA-4 Binding Agent therapy is ipilimumab.

9. The method of claim 5, wherein the Non-Responder Patient did not respond to a PD-1 Axis Binding Agent therapy.

10. (canceled)

11. The method of claim 9, wherein the PD-1 Axis Binding Agent therapy is selected from pembrolizumab, nivolumab, cemiplimab, pidilizumab, spartalizumab, atezolizumab, avelumab, and durvalumab.

12. (canceled)

13. (canceled)

14. The method of claim 1, wherein the Non-Responder Patient does not respond to an immunotherapy.

15. The method of claim 14, wherein the immunotherapy is a cancer vaccine therapy.

16. The method of claim 14, wherein the immunotherapy is T-VEC.

17. The method of claim 1, wherein the NeoTCR Product comprises CD8+ and/or CD4+ T cells from the Non-Responder Patient, wherein the T cells have been engineered to express at least one neoTCR.

18. (canceled)

19. The method of claim 17, wherein the T cells in the NeoTCR Product have been engineered to express one neoTCR.

20. The method of claim 17, wherein the T cells in the NeoTCR Product have been engineered to express a first neoTCR and a second neoTCR, wherein each T cell expresses either the first neoTCR or the second neoTCR.

21. (canceled)

22. The method of claim 17, wherein each neoTCR binds a neoepitope comprising an amino acid mutation resulting from a somatic coding mutation present in the patient's cancer.

23. The method of claim 17, wherein the neoTCR in the NeoTCR Product is present in the T cell genome at the endogenous TCR locus.

24. The method of claim 23, wherein the NeoTCR Product is produced by a non-viral engineering method.

25. (canceled)

26. The method of claim 23, wherein the NeoTCR Product does not comprise any exogenous DNA sequences in the genome of the T cells.

27. The method of claim 23, wherein the T cells of the NeoTCR Product are derived from T cells from the patient.

28. to 50. (canceled)

51. A composition for the treatment of cancer in a Non-Responder Patient comprising a polynucleotide, wherein the polynucleotide comprises:
a) first and second homology arms homologous to first and second target nucleic acid sequences;
b) a TCR gene sequence positioned between the first and second homology arms;
c) a first P2A-coding sequence positioned upstream of the TCR gene sequence and a second P2A-coding sequence positioned downstream of the TCR gene sequence, wherein the first and second P2A-coding sequences code for the same amino acid sequence that are codon-diverged relative to each other;
d) a sequence coding for a flexible linker positioned immediately upstream of the P2A-coding sequences; and
e) a sequence coding for a protease cleavage sequence positioned upstream of the second P2A-coding sequence.

52. (canceled)

53. The method of claim 1, comprising:
a) administering to a human patient a therapeutically effective population of modified primary cells, wherein the modified primary cells comprise an exogenous nuclease and a non-viral exogenous nucleic acid sequence incorporated into an endogenous locus, the non-viral exogenous nucleic acid comprising:
i. a TCR gene sequence coding for at least a portion of a TCR capable of binding an antigen present on a cancerous cell,
ii. a first P2A-coding sequence positioned upstream of the TCR gene sequence and a second P2A-coding sequence positioned downstream of the TCR gene sequence, wherein the first and second P2A-coding sequences code for the same amino acid sequence that are codon-diverged relative to each other,
iii. a sequence coding for the amino acid sequence Gly Ser Gly positioned immediately upstream of the P2A-coding sequences;
iv. a sequence coding for a Furin cleavage site positioned upstream of the second P2A-coding sequence; and
thereby treating the cancer in the human patient.

54. (canceled)

55. (canceled)

56. (canceled)

57. (canceled)

58. The method of claim 53, wherein prior to administering to the human patient a therapeutically effective population of modified primary cells, a non-myeloablative lymphodepletion regimen is administered to the patient.

59. The method of claim 53, wherein the cancer is selected from melanoma, lung cancer, breast cancer, head and neck cancer, ovarian cancer, prostate, and colorectal cancer.

60. The method of claim 1, comprising:
a) modifying a patient-derived T cell by introducing a nuclease-mediated introduction of a non-viral polynucleotide into the T cell, wherein the non-viral polynucleotide comprises:
i. first and second homology arms homologous to first and second endogenous sequences of the cell;
ii. a TCR gene sequence positioned between the first and second homology arms; and
iii. a first P2A-coding sequence positioned upstream of the TCR gene sequence and a second P2A-coding sequence positioned downstream of the TCR gene sequence, wherein the first and second P2A-coding sequences code for the same amino acid sequence that are but codon-diverged relative to each other;
iv. a sequence coding for the amino acid sequence Gly Ser Gly positioned immediately upstream of the P2A-coding sequences; and
v. a sequence coding for a Furin cleavage site positioned upstream of the second P2A-coding sequence;
b) recombining the non-viral polynucleotide into an endogenous locus of the T cell, wherein the endogenous locus comprises the first and second endogenous sequences homologous to the first and second homology arms of the non-viral polynucleotide; and
c) culturing the modified T cell to produce a population of T cells; and
d) administering a therapeutically effective number of the modified T cells to the human patient to thereby treat the cancer.

61. to 71. (canceled)
